(19) United States
(12) Patent Application Publication
(10) Pub. No.: US 2010/0048641 A1
(43) Pub. Date: Feb. 25, 2010

(54) Use of Adenosine A1 and/or Dual A1/2ab Agonists for Production of Medicaments for Treating Diseases

(75) Inventors: Peter Nell, Wuppertal (DE); Barbara Albrecht-Küpper, Wulfrath (DE); Walter Hübsch, Wuppertal (DE); Martina Wuttke, Leichlingen (DE); Thomas Krahn, Hagen (DE); Nicole Diedrichs, Velbert (DE); Hilmar Bischoff, Wuppertal (DE)

Correspondence Address: Barbara A. Shimei;Director, Patents & Licensing, Bayer HealthCare LLC - Pharmaceuticals, 555 White Plains Road, Third Floor, Tarrytown, NY, 10591, US

(73) Assignee: BAYER HEALTHCARE AG LAW AND PATENTS,PATENTS AND LICENSING, LEVERKUSEN (DE)

(21) Appl. No.: 12/224,417

(22) PCT Filed: Feb. 16, 2007
(86) PCT No.: PCT/EP2007/001342

§ 371 Date: Aug. 5, 2009

(30) Foreign Application Priority Data
Mar. 1, 2006 (DE) 10 2006 009 813,7

(51) Int. Cl. A61K 31/4439; C07D 417/12; A61P 3/00; A61P 3/10

(52) U.S. Cl. 514/342; 546/270.7

(57) ABSTRACT

The present invention relates to the use of A1 and/or dual A1/A2b agonists of the formulae (IA) and (IB) for preparing a medicament for treating dyslipidemia, metabolic syndrome and diabetes and dyslipidemia, metabolic syndrome and diabetes in association with hypertension and cardiovascular disorders.

The present invention relates to the use of A1 and/or dual A1/A2b agonists of the formulae (IA) and (IB) for preparing a medicament for treating dyslipidemia, metabolic syndrome and diabetes and dyslipidemia, metabolic syndrome and diabetes in association with hypertension and cardiovascular disorders.

More than 180 million people in the USA, Europe and Japan are suffering from hypertension. With the population getting progressively older, this proportion will increase even further. 65% of all patients having diagnosed hypertension also suffer from dyslipidemia, and 16% from diabetes. An even higher percentage is suffering from an early stage of these disorders, the metabolic syndrome. Owing to these additional illnesses, these patients have a greatly increased risk of developing cardiovascular disorders, such as CHD, angina pectoris, arteriosclerosis and myocardial insufficiency. In spite of many successful therapies, cardiovascular disorders remain a serious public health problem. Treatment of these high-risk patients with a drug which would not only reduce blood pressure and/or heart rate but also have a positive effect on these additional disorders would be of great benefit for the patient.

Adenosine, a purine nucleoside, is present in all cells and is released under a large number of physiological and pathophysiological stimuli. Adenosine is produced inside cells on degradation of adenosine 5′-monophosphate (AMP) and S-adenosylhomocysteine as intermediate, but can be released from the cell and then exerts, by binding to specific receptors, effects as a hormone-like substance or neurotransmitter.

Under normoxic conditions, the concentration of free adenosine in the extracellular space is very low. However, under ischemic or hypoxic conditions, the extracellular concentration of adenosine in the affected organs increases dramatically. Thus, it is known, for example, that adenosine inhibits platelet aggregation and increases coronary perfusion. It also acts on blood pressure, heart rate, on the secretion of neurotransmitters and on lymphocyte differentiation.

In adipocytes, adenosine is capable of inhibiting lypolysis via activation of specific adenosine receptors, thus lowering the concentration of free fatty acids and triglycerides in the blood.

Hitherto, it is known that the action of adenosine is mediated via four specific receptors. To date the subtypes A1, A2a, A2b and A3 are known. The actions of these adenosine receptors are mediated intracellularly by the messenger cAMP. In the case of binding of adenosine to the A2a or A2b receptors, the intracellular cAMP is increased via activation of the membrane-bound adenylate cyclase, whereas binding of adenosine to the A1 or A3 receptors results in the intracellular cAMP concentration being kept low via inhibition of adenylate cyclase.

In the cardiovascular system, the main consequences of the activation of adenosine receptors are: bradycardia, negative inotropism and protection of the heart against ischemia (“preconditioning”) via A1 receptors, dilation of the blood vessels via A2a and A2b receptors and inhibition of the fibroblasts and smooth-muscle-cell proliferation and migration via A2b receptors.

The activation of A2b receptors by adenosine or specific A2b agonists leads, via dilation of blood vessels, to lowering of the blood pressure. The lowering of the blood pressure is frequently accompanied by a refectory increase in heart rate.

Tachycardia or a refectory increase in heart rate can be treated or reduced by activation of A1 receptors using specific A1 agonists.

The combined action of selective A1/A2b agonists on the vascular system and the heart rate thus results in a systemic lowering of the blood pressure with significantly reduced tendency toward a reflectory heart-rate increase. Dual A1/A2b agonists having such a pharmacological profile could be employed, for example, for treating hypertension in humans.

In adipocytes, the activation of A1 and A2b receptors leads to an inhibition of lypolysis. Thus, the individual or else the combined action of A1 or A1/A2b agonists on lipid metabolism results in a lowering of free fatty acids and/or triglycerides. In turn, for example in patients suffering from metabolic syndrome and in diabetics, a lowering of the lipids or the free fatty acids leads to lower insulin resistance and improved symptoms.

The ligands known from the prior art, which are referred to as “adenosine receptor-specific” are mainly derivatives based on natural adenosine [S.-A. Poulsen and R. J. Quinn, “Adenosine receptors: new opportunities for future drugs” in Bioorganic and Medicinal Chemistry 6 (1998), pages 619-641]. However, most of these adenosine ligands known from the prior art have the disadvantage that their action is not really receptor-specific, that their activity is less than that of natural adenosine or that they have only very weak activity after oral administration. Thus, they are mainly used only for experimental purposes.

WO 02/06237 discloses aryl-substituted dicyanopyridines as calcium-dependent potassium channel openers and their use in the treatment of disorders of the urogenital tract. Furthermore, WO 01/25210 and WO 02/070485 describe substituted 2-thio-3,5-dicyano-4-aryl-6-aminopyridines as adenosine receptor ligands for the treatment of disorders. WO 03/053441 discloses specifically substituted 2-thio-3,5-dicyano-4-phenyl-6-aminopyridines as selective ligands of the adenosine A1 receptor for the treatment of in particular cardiovascular disorders. WO 02/50071 describes aminothiazole derivatives as tyrosine kinase inhibitors for the treatment of various illnesses.

Accordingly, it is an object of the present invention to provide compounds which act as selective agonists of the adenosine A1 receptor or as selective dual agonists of the A1/A2b receptor and are, as such, suitable for the treatment and/or the prevention of dyslipidemia, metabolic syndrome and diabetes.

It is another object of the invention to provide compounds which act as selective agonists of the adenosine A1 receptor or as selective dual agonists of the A1/A2b receptor and, as such, are suitable for the treatment and/or the prevention of dyslipidemia, metabolic syndrome and diabetes in association with hypertension and cardiovascular disorders.

A further object of the invention is the provision of compounds which, in combination act as selective agonists of the adenosine A1 receptor and as selective dual agonists of the A1/A2b receptor and, as such, are suitable for the treatment and/or the prevention of dyslipidemia, metabolic syndrome and diabetes and dyslipidemia, metabolic syndrome and diabetes in association with hypertension and cardiovascular disorders.

The present invention provides the use of compounds of the formula (IA)

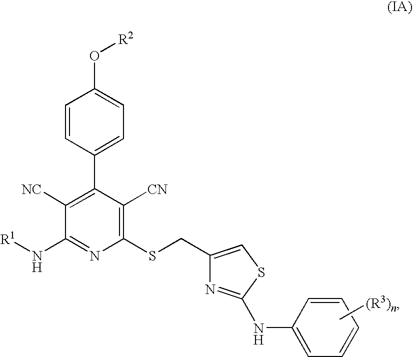

in which
R¹ represents hydrogen or represents (C₁-C₆)-alkyl which may be substituted by hydroxyl, amino, mono- or di-(C₁-C₄)-alkylamino, pyrrolidino, piperidino, morpholino, piperazino or N′-methylpiperazino,
R² represents (C₂-C₆)-alkyl, which is mono- or di-substituted by identical or different substituents selected from the group consisting of hydroxyl, (C₁-C₄)-alkoxy, amino, mono- and di-(C₁-C₄)-alkylamino,
R³ represents a substituent selected from the group consisting of halogen, cyano, nitro, (C₁-C₆)-alkyl, hydroxyl, (C₁-C₆)-alkoxy, amino, mono- and di-(C₁-C₆)-alkylamino, carboxyl and (C₁-C₆)-alkoxycarbonyl,
   where alkyl and alkoxy for their part may each be substituted up to five times by fluorine,
   and
n represents the number 0, 1, 2, 3, 4 or 5,
   where, if the substituent R³ is present more than once, its meanings may be identical or different,

and their salts, solvates and solvates of the salts for preparing a medicament for treating dyslipidemia, metabolic syndrome and diabetes.

Compounds according to the invention are the compounds of the formulae (IA) and (IB) and their salts, solvates and solvates of the salts, the compounds of the formulae mentioned below or in WO 03/053441 embraced by the formulae (IA) and (IB) and their salts, solvates and solvates of the salts, and the compounds mentioned below or in WO 03/053441 as working examples embraced by the formulae (IA) and (IB) and their salts, solvates and solvates of the salts, provided the compounds mentioned below or in WO 03/053441 embraced by the formulae (IA) and (IB) are not already salts, solvates and solvates of the salts.

The compounds of the formulae (IA) and (IB) according to the invention may, depending on their structure, exist in stereoisomeric forms (enantiomers, diastereomers). The invention therefore embraces the enantiomers or diastereomers and respective mixtures thereof. The stereoisomerically pure constituents can be isolated from such mixtures of enantiomers and/or diastereomers in a known manner.

Where the compounds according to the invention can exist in tautomeric forms, the present invention embraces all tautomeric forms.

Salts preferred for the purposes of the present invention are physiologically acceptable salts of the compounds according to the invention. Also included are salts which are not themselves suitable for pharmaceutical applications but can be used, for example, for the isolation or purification of the compounds according to the invention.

Physiologically acceptable salts of the compounds according to the invention include acid addition salts of mineral acids, carboxylic acids and sulfonic acids, for example salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, trifluoroacetic acid, propionic acid, lactic acid, tartaric acid, malic acid, citric acid, fumaric acid, maleic acid and benzoic acid.

Physiologically acceptable salts of the compounds according to the invention also include salts of conventional bases such as, by way of example and by way of preference, alkali metal salts (for example sodium and potassium salts), alkaline-earth metal salts (for example calcium and magnesium salts) and ammonium salts, derived from ammonia or organic amines having 1 to 16 carbon atoms, such as by way of example and by way of preference ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

Solvates refer for the purposes of the invention to those forms of the compounds according to the invention which form a complex in the solid or liquid state through coordination with solvent molecules. Hydrates are a specific form of solvates in which the coordination takes place with water. For the purposes of the present invention, preferred solvates are hydrates.

In addition, the present invention also embraces prodrugs of the compounds according to the invention. The term “prodrugs” embraces compounds which for their part may be biologically active or inactive but are converted (for example metabolically or hydrolytically) into compounds according to the invention during their residence time in the body.

For the purposes of the present invention, the substituents have the following meaning, unless specified otherwise:

For the purposes of the invention, (C₁-C₆)-alkyl, (C₂-C₆)-alkyl, (C₁-C₄)-alkyl and (C₂-C₄)-alkyl are straight-chain or branched alkyl radicals having 1 to 6, 2 to 6, 1 to 4 and 2 to 4 carbon atoms, respectively. Preference is given to a straight-chain or branched alkyl radical having 1 to 4 or 2 to 4 carbon atoms. The following radicals may be mentioned by way of example and by way of preference: methyl, ethyl, n-propyl, isopropyl, n-butyl, isobutyl, sec-butyl, tert-butyl, 1-ethylpropyl, n-pentyl and n-hexyl.

For the purposes of the invention, (C₁-C₆)-alkoxy and (C₁-C₄)-alkoxy are straight-chain or branched alkoxy radicals having 1 to 6 and 1 to 4 carbon atoms, respectively. Preference is given to a straight-chain or branched alkoxy radical having 1 to 4 carbon atoms. The following radicals may be mentioned by way of example and by way of preference: methoxy, ethoxy, n-propoxy, isopropoxy and tert-butoxy.

For the purposes of the invention, (C₁-C₆)-alkoxycarbonyl and (C₁-C₄)-alkoxycarbonyl are straight-chain or branched alkoxy radicals having 1 to 6 and 1 to 4 carbon atoms, respectively, which are attached via a carbonyl group. Preference is given to a straight-chain or branched alkoxycarbonyl radical having 1 to 4 carbon atoms in the alkoxy group. The following radicals may be mentioned by way of example and by way of preference: methoxycarbonyl, ethoxycarbonyl, n-propoxycarbonyl, isopropoxycarbonyl and tert-butoxycarbonyl.

For the purposes of the invention, mono-(C₁-C₆)-alkylamino and mono-(C₁-C₄)-alkylamino are amino groups having a straight-chain or branched alkyl substituent, having 1 to 6 and 1 to 4 carbon atoms, respectively. Preference is given to a straight-chain or branched monoalkylamino radical having 1 to 4 carbon atoms. The following radicals may be mentioned by way of example and by way of preference: methylamino, ethylamino, n-propylamino, isopropylamino and tert-butylamino.

For the purposes of the invention, di-(C₁-C₆)-alkylamino and di-(C₁-C₄)-alkylamino are amino groups having two identical or different straight-chain or branched alkyl substituents, having 1 to 6 and 1 to 4 carbon atoms, respectively. Preference is given to straight-chain or branched dialkylamino radicals having in each case 1 to 4 carbon atoms. The following radicals may be mentioned by way of example and by way of preference: N,N-dimethylamino, N,N-diethylamino, N-ethyl-N-methylamino, N-methyl-N-n-propylamino, N-isopropyl-N-n-propylamino, N-tert-butyl-N-methylamino, N-ethyl-N-n-pentylamino and N-n-hexyl-N-methylamino.

For the purposes of the invention halogen includes fluorine, chlorine, bromine and iodine. Preference is given to chlorine or fluorine.

When radicals in the compounds according to the invention are substituted, the radicals may be mono- or poly-substituted unless specified otherwise. For the purposes of the present invention, the meanings of all radicals which occur more than once are independent of one another. Preference is given to substitution by one, two or three identical or different substituents. Very particularly preferred is substitution by one or two identical or different substituents.

For the purpose of the present invention, preference is given to using compounds of the formula (IA),

in which
R¹ represents hydrogen or represents (C₁-C₄)-alkyl, which may be substituted by hydroxyl, amino or dimethylamino,
R² represents (C₂-C₄)-alkyl, which is mono- or disubstituted by identical or different substituents selected from the group consisting of hydroxyl, methoxy and amino,
R³ represents a substituent selected from the group consisting of halogen, cyano, nitro, (C₁-C₄)-alkyl, hydroxyl, (C₁-C₄)-alkoxy, amino, mono- and di-(C₁-C₄)-alkylamino, carboxyl and (C₁-C₄)-alkoxycarbonyl,
   where alkyl and alkoxy for their part may each be substituted up to three times by fluorine,

and
n represents the numbers 0, 1 or 2,
   where, if the substituent R³ is present twice, its meanings may be identical or different,

and their salts, solvates and solvates of the salts for preparing a medicament for treating dyslipidemia, metabolic syndrome and diabetes.

For the purposes of the present invention, particular preference is given to using compounds of the formula (IA),

in which
R¹ represents hydrogen,
R² represents ethyl, n-propyl or isopropyl, which are in each case mono- or disubstituted by identical or different substituents selected from the group consisting of hydroxyl, methoxy and amino,
R³ represents a substituent selected from the group consisting of fluorine, chlorine, bromine, cyano, nitro, methyl, ethyl, trifluoromethyl, hydroxyl, methoxy, ethoxy, amino, mono- and dimethylamino, carboxyl, methoxycarbonyl and ethoxycarbonyl,

and
n represents the numbers 0, 1 or 2,
   where, if the substituent R³ is present twice, its meanings may be identical or different, and their salts, solvates and solvates of the salts for preparing a medicament for treating dyslipidemia, metabolic syndrome and diabetes.

The compounds of the formula (IA) can be prepared by the following process.

Process, characterized in that compounds of the formula (II)

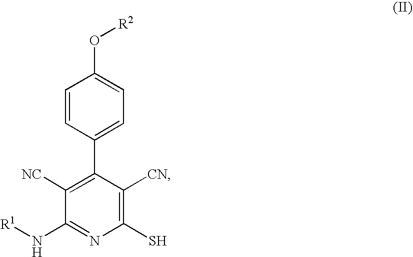

in which R¹ and R² each have the meanings given above,

are reacted in an inert solvent in the presence of a base with a compound of the formula (III)

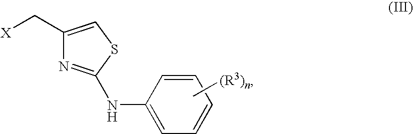

in which R³ and n have the meanings given above and
X represents a suitable leaving group, preferably halogen, in particular chlorine, bromine or iodine, or represents mesylate, tosylate or triflate,

and the compounds of the formula (IA) are, if appropriate, converted with the appropriate (i) solvents and/or (ii) bases or acids into their solvates, salts and/or solvates of the salts.

The process described above can be illustrated in an exemplary manner by the formula scheme below:

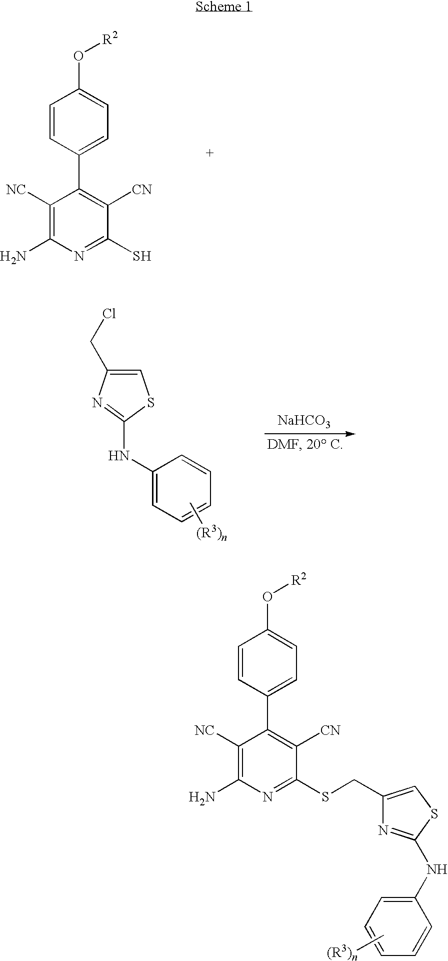

Suitable solvents for the process according to the invention are all organic solvents which are inert under the reaction conditions. These include alcohols such as methanol, ethanol and isopropanol, ketones such as acetone and methylethyl ketone, acyclic and cyclic ethers such as diethylether, tetrahydrofuran and dioxane, esters such as ethyl acetate or butyl acetate, hydrocarbons, such as benzene, toluene, xylene, hexane or cyclohexane, chlorinated hydrocarbons such as dichloromethane or chlorobenzene, or other solvents, such as dimethylformamide, acetonitrile, pyridine or dimethyl sulfoxide. Another suitable solvent is water. It is also possible to use mixtures of the solvents mentioned above. A preferred solvent is dimethylformamide.

Suitable bases are the customary inorganic or organic base. These preferably include alkali metal hydroxides, such as, for example sodium hydroxide or potassium hydroxide, alkali metal carbonates such as sodium carbonate, potassium carbonate or cesium carbonate, alkali metal bicarbonates such as sodium bicarbonate or potassium bicarbonate, alkali metal alkoxides, such as sodium methoxide or potassium methoxide, sodium ethoxide or potassium ethoxide or potassium tert-butoxide, or amides such as sodium amide, lithium bis(trimethylsilyl)amide or lithium diisopropylamide, or organometallic compounds, such as butyllithium or phenyllithium, or organic amines such as triethylamine, pyridine, 1,8-diazabicyclo[5.4.0]undec-7-ene (DBU) or 1,5-diazabicyclo[4.3.0]non-5-ene (DBN). Preference is given to alkali metal carbonates and bicarbonates.

Here, the base can be employed in an amount of from 1 to 10 mol, preferably from 1 to 5 mol, in particular from 1 to 4 mol, based on 1 mol of the compound of the formula (II).

The reaction is generally carried out in a temperature range of from −78° C. to +140° C., preferably in the range from −20° C. to +60° C., in particular at from 0° C. to +40° C. The reaction can be carried out at atmospheric, elevated or reduced pressure (for example in the range of from 0.5 to 5 bar). In general the reaction is carried out at atmospheric pressure.

Compounds of the formula (II), in which R¹ represents hydrogen are known per se to the person skilled in the art or can be prepared by customary methods known from the literature. Reference may be made, in particular, to applications below, whose respective content is incorporated herein by way of reference:
a) Dyachenko et al., Russian Journal of Chemistry 33 (7), 1014-1017 (1997) and 34 (4), 557-563 (1998);
b) Dyachenko et al., Chemistry of Heterocyclic Compounds 34 (2), 188-194 (1998);
c) Qintela et al., European Journal of Medicinal Chemistry 33, 887-897 (1998);
d) Kandeel et al., Zeitschrift für Naturforschung 42b, 107-111 (1987).

The compounds of the formula (II), in which R¹ represents hydrogen, can also be prepared from compounds of the formula (IV)

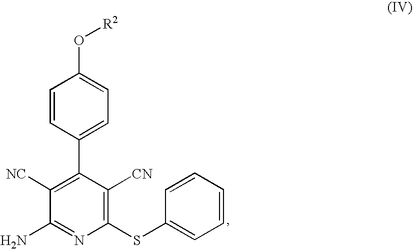

in which R² has the meaning given above,

by reaction with an alkali metal sulfide. This preparation method can be illustrated in an exemplary manner by the formula scheme below:

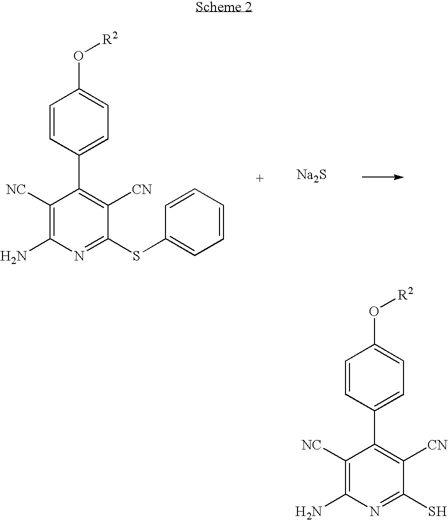

The alkali metal sulfide used is preferably sodium sulfide in an amount of from 1 to 10 mol, preferably from 1 to 5 mol, in particular from 1 to 4 mol, based on 1 mol of the compound of the formula (IV).

Suitable solvents are all organic solvents which are inert under the reaction conditions. These preferably include N,N-dimethylformamide, N-methylpyrrolidinone, pyridine and acetonitrile. It is also possible to use mixtures of the solvents mentioned above. Particular preference is given to N,N-dimethylformamide.

The reaction is generally carried out in a temperature range of from +20° C. to +140° C., preferably in a range of from +20° C. to +120° C., in particular at from +60° C. to +100° C. The reaction can be carried out at atmospheric, elevated or reduced pressure (for example in the range of from 0.5 to 5 bar). In general, the reaction is carried out at atmospheric pressure.

The compounds of the formula (IV) can be prepared analogously to the compounds described in the publications below:

a) Kambe et al., Synthesis, 531-533 (1981);

b) Elnagdi et al., Z. Naturforsch. 47b, 572-578 (1991).

Compounds of the formula (II), in which R¹ does not represent hydrogen, can be prepared by converting compounds of the formula (IV) initially with copper(II) chloride and isoamyl nitrite in a suitable solvent and compounds of the formula (V)

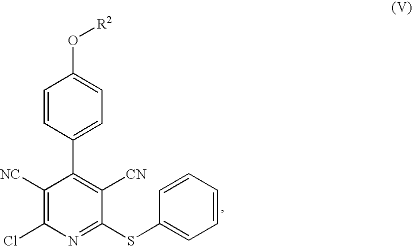

in which R² has the meaning given above,

then reacting these with a compound of the formula (VI)

R^{1A}—NH₂ (VI),

in which

R^{1A} has the meaning of R¹ given above, but does not represent hydrogen,

to give compounds of the formula (VII)

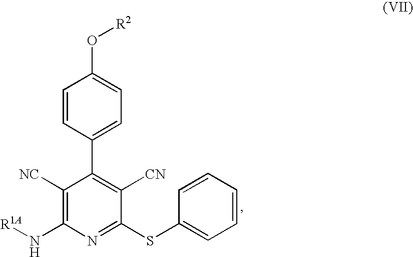

in which R^{1A} and R² each have the meanings given above,

and then converting it with sodium sulfide into compounds of the formula (II).

The process described above can be illustrated in an exemplary manner by the formula scheme below:

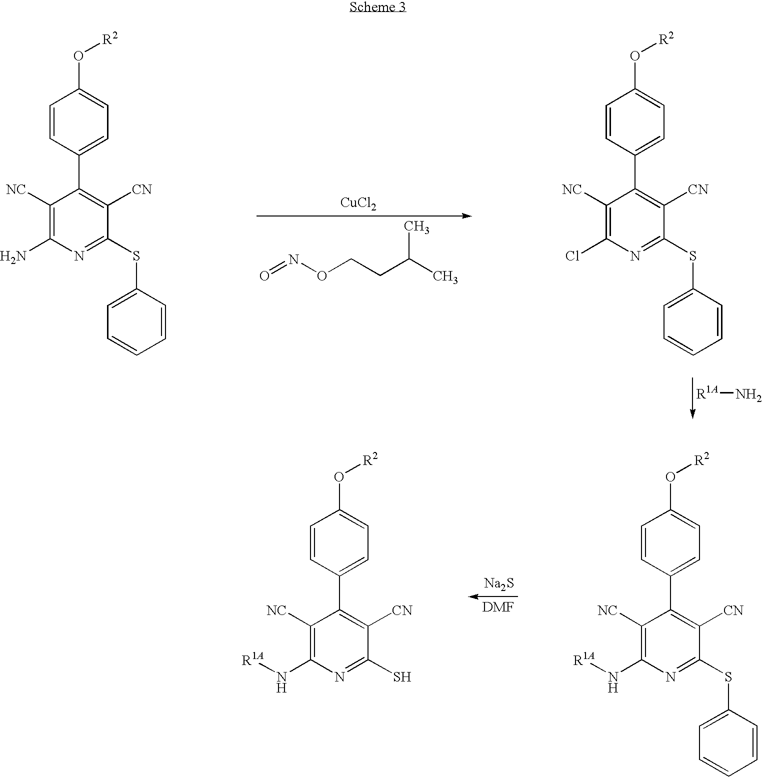

The process step (IV)→(V) is generally carried out using a molar ratio of from 2 to 12 mol of copper(II) chloride and from 2 to 12 mol of isoamyl nitrite, based on 1 mol of the compound of the formula (IV).

Suitable solvents for this process step are all organic solvents which are inert under the reaction conditions. These include acyclic and cyclic ethers such as diethyl ether and tetrahydrofuran, esters such as ethyl acetate or butyl acetate, hydrocarbons such as benzene, toluene, xylene, hexane or cyclohexane, chlorinated hydrocarbons such as dichloromethane, dichloroethane or chlorobenzene, or other solvents, such as dimethylformamide, acetonitrile or pyridine. It is also possible to use mixtures of the solvents mentioned above. Preferred solvents are acetonitrile and dimethylformamide.

The reaction is generally carried out in a temperature range of from −78° C. to +180° C., preferably in the range from +20° C. to +100° C., in particular at from +20° C. to +60° C. The reaction can be carried out at atmospheric, elevated or reduced pressure (for example in the range of from 0.5 to 5 bar). In general, the reaction is carried out at atmospheric pressure.

The process step (V)+(VI)→(VIII) is generally carried out using a molar ratio of from 1 to 8 mol of the compound of the formula (VI), based on 1 mol of the compound of the formula (V).

Suitable solvents for this reaction step are all organic solvents which are inert under the reaction conditions. These include alcohols, such as methanol, ethanol and isopropanol, ketones, such as acetone and methyl ethyl ketone, acyclic and cyclic ethers such as diethyl ether and tetrahydrofuran, esters, such as ethyl acetate or butyl acetate, hydrocarbons such as benzene, toluene, xylene, hexane or cyclohexane, chlorinated hydrocarbons such as dichloromethane, dichloroethane or chlorobenzene, or other solvents, such as dimethylformamide, acetonitrile, pyridine or dimethyl sulfoxide. Another suitable solvent is water. It is also possible to use mixtures of the solvents mentioned above. A preferred solvent is dimethylformamide.

The reaction is generally carried out in a temperature range of from −78° C. to +180° C., preferably in the range of from +20° C. to +160° C., in particular at from +20° C. to +40° C. The reaction can be carried out at atmospheric, elevated or reduced pressure (for example in the range of from 0.5 to 5 bar). In general, the reaction is carried out at atmospheric pressure.

The process step (VII)→(II) is generally carried out using a molar ratio of from 1 to 8 mol of sodium sulfide, based on 1 mol of the compound of the formula (VII).

Suitable solvents for this reaction step are all organic solvents which are inert under the reaction conditions. These include alcohols, such as methanol, ethanol and isopropanol, ketones, such as acetone and methyl ethyl ketone, acyclic and cyclic ethers such as diethyl ether and tetrahydrofuran, esters, such as ethyl acetate or butyl acetate, hydrocarbons such as benzene, toluene, xylene, hexane or cyclohexane, chlorinated hydrocarbons such as dichloromethane, dichloroethane or chlorobenzene, or other solvents, such as dimethylformamide, acetonitrile, pyridine or dimethyl sulfoxide. It is also possible to use mixtures of the solvents mentioned above. A preferred solvent is dimethyl formamide.

The reaction is generally carried out in a temperature range of from −78° C. to +180° C., preferably in the range of from +20° C. to +160° C., in particular at from +40° C. to +100° C. The reaction can be carried out at atmospheric, elevated or reduced pressure (for example in the range of from 0.5 to 5 bar). In general, the reaction is carried out at atmospheric pressure.

The compounds of the formula (VI) are either commercially available, known to the person skilled in the art or preparable by customary methods.

Compounds of the formula (III) can be prepared from compounds of the formula (VIII)

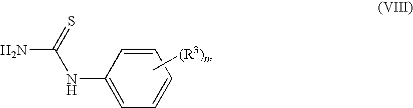

in which R³ and n have the meanings given above,

by reaction with a 1,3-dihaloacetone. This preparation method can be illustrated in exemplary manner by the formula scheme below:

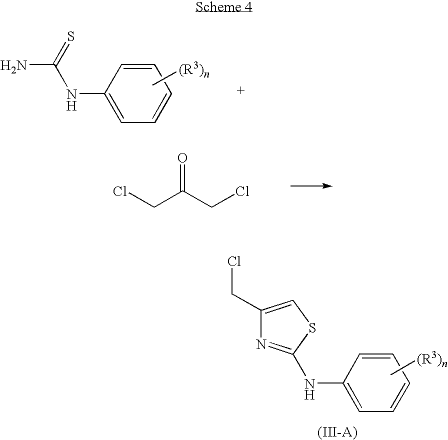

Here, the compounds of the formula (III-A) can either be prepared and isolated analogously to the literature [I. Simiti et al., Chem. Ber. 95, 2672-2679 (1962)] or they can be generated in situ and directly reacted further with a compound of the formula (II). Preferance is given to the in situ generation from 1,3-dichloracetone and a compound of the formula (VIII) in dimethylformamide or ethanol. The preparation is generally carried out in a temperature range of from 0° C. to +140° C., preferably in the range of from +20° C. to +120° C., in particular at from +80° C. to +100° C.

The compounds of the formula (VIII) are either commercially available, known to the person skilled in the art or preparable by customary methods.

The present invention also provides the use of compounds of the formula (IB)

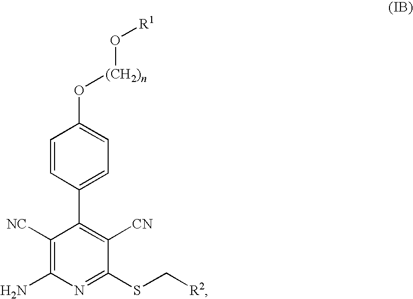

in which
n represents a number 2, 3 or 4,
R¹ represents hydrogen or (C₁-C₄)-alkyl

and
R² represents pyridyl or thiazolyl, which for its part may be substituted by (C₁-C₄)-alkyl, halogen, amino, dimethylamino, acetylamino, guanidino, pyridylamino, thienyl, furyl, imidazolyl, pyridyl, morpholinyl, thiomorpholinyl, piperidinyl, piperazinyl, N—(C₁-C₄)-alkylpiperazinyl, pyrrolidinyl, oxazolyl, isoxazolyl, pyrimidinyl, pyrazinyl, optionally (C₁-C₄)-alkyl-substituted thiazolyl or phenyl which is optionally substituted up to three times by halogen, (C₁-C₄)-alkyl or (C₁-C₄)-alkoxy,

and their salts, hydrates hydrates of the salts and solvates for preparing a medicament for treating dyslipidemia, metabolic syndrome and diabetes.

Also preferred is the use of compounds of the formula (IB)

in which
n represents the number 2,
R¹ represents hydrogen, methyl or ethyl

and
R² represents pyridyl or thiazolyl which for its part may be substituted by methyl, ethyl, fluorine, chlorine, amino, dimethylamino, acetylamino, guanidino, 2-pyridylamino, 4-pyridylamino, thienyl, pyridyl, morpholinyl, piperidinyl, optionally methyl-substituted thiazolyl or phenyl substituted up to three times by chlorine or methoxy,

and their salts, hydrates, hydrates of the salts and solvates for preparing a medicament for treating dyslipidemia, metabolic syndrome and diabetes.

Particular preference is given to using compounds of the formula (IB), in which R¹ represents hydrogen or methyl for preparing a medicament for treating dyslipidemia, metabolic syndrome and diabetes.

Particular preference is also given to using compounds of the formula (IB), in which
n represents the number 2,
R¹ represents hydrogen or methyl

and
R² represents pyridyl or thiazolyl, which for its part may be substituted by methyl, chlorine, amino, dimethylamino, acetylamino, guanidino, 2-pyridylamino, 4-pyridylamino, thienyl, pyridyl, morpholinyl, 2-methylthiazol-5-yl, phenyl, 4-chlorophenyl or 3,4,5-trimethoxyphenyl,

and their salts, hydrates, hydrates of the salts and solvates for preparing a medicament for treating dyslipidemia, metabolic syndrome and diabetes.

Very particular preference is given to using the compound from example 6 of WO 03/053441 having the structure below

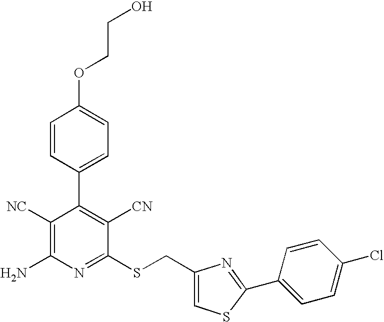

and its salts, hydrates, hydrates of the salts and solvates for preparing a medicament for treating dyslipidemia, metabolic syndrome and diabetes.

The compounds of the formula (IB), their preparation and explicitly mentioned examples are known from WO 03/053441. The teaching of WO 03/053441 is hereby expressly incorporated into this disclosure.

The invention furthermore preferably provides the use of compounds of the formulae (IA) and (IB), their salts, solvates and solvates of the salts for preparing a medicament for the treatment of dyslipidemia, metabolic syndrome and diabetes.

The invention furthermore preferably provides the use of compounds of the formulae (IA) and/or (IB), their salts, solvates and solvates of the salts for preparing a medicament for treating dyslipidemia, metabolic syndrome and diabetes in association with hypertension and cardiovascular disorders.

The compounds of the formula (IA) have been found to be dual agonists of adenosine acting selectively at A1 and A2b receptors.

The compounds of the formula (IB) have been found to be single agonists of adenosine acting selectively at the A1 receptors.

Specific A1 agonists differ from the corresponding dual A1/A2b agonists in that specific A1 agonists have an agonistic effect on the A1 receptor which, compared to the effect on the A2b receptor of the respective same species, is greater by a factor of ≧10. The specificity can be determined in appropriate in vitro assays based on the concentration and/or in in vivo experiments based on the respective dosage.

Surprisingly, the compounds of the formulae (IA) and (IB) according to the invention have an unforeseeable useful pharmacological activity spectrum and are thus particularly suitable for the prophylaxis and/or treatment of dyslipidemia, metabolic syndrome and diabetes and dyslipidemia, metabolic syndrome and diabetes in association with hypertension and cardiovascular disorders and for preparing a medicament for treating dyslipidemia, metabolic syndrome and diabetes and dyslipidemia, metabolic syndrome and diabetes in association with hypertension and cardiovascular disorders.

The pharmaceutical activity of the compounds according to the invention can be explained by their action as selective ligands at adenosine A1 and A2b receptors. Here, compounds of the formula (IB) act as single A1 agonists and the compounds of the formula (IA) act as dual A1/A2b agonists.

According to the invention, “adenosine receptor-selective ligands” are those substances which bind selectively to one or more subtypes of the adenosine receptors, thus either mimicking the action of adenosine (adenosine agonists) or blocking its action (adenosine antagonists).

In the context of the present invention, “selective” are those adenosine receptor ligands where, firstly, a marked activity at A1 or A1/A2b adenosine receptor subtypes can be observed and, secondly, no or a considerably weaker activity (factor 10 or more) at A2a and A3 adenosine receptor subtypes can be observed.

For the purpose of the present invention, disorders of the cardiovascular system or cardiovascular disorders are to be understood as meaning, in addition to hypertension, in particular the following disorders: coronary restenosis, such as, for example, restenosis after balloon dilation of peripheral blood vessels, tachycardias, arrhythmias, disorders of peripheral and cardial blood vessels, stable and unstable angina pectoris, atrial and ventrial fibrillation and myocardial insufficiency.

The present invention furthermore relates to a method for the prophylaxis and/or treatment of the syndromes mentioned above using the compounds of the formulae (IA) and (IB).

The present invention furthermore provides medicaments comprising at least one compound according to the invention, usually together with one or more inert nontoxic pharmaceutically suitable auxiliaries, and their use for the purposes mentioned above.

The compounds according to the invention can act systemically and/or locally. For this purpose, they can be administered in a suitable manner, such as, for example, orally, parenterally, pulmonarily, nasally, sublingually, lingually, buccally, rectally, dermally, transdermally, conjunctivally, otically or as an implant or stent.

For these administration routes, the compounds according to the invention can be administered in suitable administration forms.

Suitable for oral administration are administration forms which work according to the prior art, deliver the compounds according to the invention rapidly and/or in modified form and which comprise the compounds according to the invention in crystalline and/or amorphisized and/or dissolved form, such as, for example, tablets (uncoated or coated tablets, for example tablets provided with enteric coatings or coatings which dissolve in a delayed manner or are insoluble and which control the release of the compound according to the invention), tablets which rapidly disintegrate in the oral cavity or films/wafers, films/lyophilizates, capsules (for example hard or soft gelatin capsules), sugar-coated tablets, granules, pellets, powders, emulsions, suspensions, aerosols or solutions.

Parenteral administration can be carried out with avoidance of an absorption step (for example intravenously, intraarterialy, intracardialy, intraspinaly or intralumbarly) or with involvement of an absorption (for example intramuscularly, subcutaneously, intracutaneously, percutaneously or intraperitonealy). Suitable administration forms for parenteral administration are, inter alia, injection and infusion preparations in the form of solutions, suspensions, emulsions, lyophilizates or sterile powders.

Suitable for the other administration routes are, for example, pharmaceutical forms for inhalation (inter alia powder inhalers, nebulizers), nasal drops, solutions or sprays, tablets to be administered lingually, sublingually or buccally, films/wafers or capsules, suppositories, aural and ophthalmic preparations, vaginal capsules, aqueous suspensions (lotions, shaker mixtures), lipophilic suspensions, ointments, creams, transdermal therapeutic systems (for example patches), milk, pastes, foams, dusting powders, implants or stents.

Preference is given to oral or parenteral administration, in particular to oral administration.

The compounds according to the invention can be converted into the administration forms mentioned. This may take place in a manner known per se by mixing with inert nontoxic pharmaceutically suitable auxiliaries. These auxiliaries include, inter alia, carriers (for example microcrystalline cellulose, lactose, mannitol), solvents (for example liquid polyethylene glycols), emulsifiers and dispersants or wetting agents (for example sodium dodecylsulfate, polyoxysorbitan oleate), binders (for example polyvinylpyrrolidone), synthetic and natural polymers (for example albumin), stabilizers (for example antioxidants, such as, for example, ascorbic acid), colorants (for example inorganic pigments, such as, for example, iron oxides) and taste and/or odor correctants.

In general, it has been found to be advantageous to administer, in the case of parenteral administration, amounts of from about 0.001 to 1 mg/kg, preferably about 0.01 to 0.5 mg/kg, of body weight to obtain effective results. In the case of oral administration, the dosage is from about 0.01 to 100 mg/kg, preferably about 0.01 to 20 mg/kg and very particularly preferably 0.1 to 10 mg/kg, of body weight.

In spite of this, it may, if appropriate, be necessary to depart from the amounts mentioned, namely depending on the body weight, the administration route, the individual response to the active compound, the type of preparation and the time or interval at which administration takes place. Thus, in some cases, it may be sufficient to manage with less than the abovementioned minimum amount, while in other cases the upper limit mentioned has to be exceeded. In the case of the administration of relatively large amounts, it may be advisable to divide these into a number of individual doses over the course of the day.

The following exemplary embodiments illustrate the invention. The invention is not limited to the examples.

The percentages in the tests and examples below are, unless indicated otherwise, percentages by weight; parts are parts by weight. Solvent ratios, dilution ratios and stated concentrations of liquid/liquid solutions are in each case based on volume.

### A. EXAMPLES

### Abbreviations Used:

Ex. example
TLC thin-layer chromatography
DCI direct chemical ionization (in MS)
DMF N,N-dimethylformamide
DMSO dimethyl sulfoxide
EA ethyl acetate
EI electron impact ionization (in MS)
ESI electrospray ionization (in MS)
m.p. melting point
sat. saturated
h hour(s)
HPLC high-pressure, high-performance liquid chromatography
conc. concentrated
LC-MS liquid chromatography-coupled mass spectroscopy
LDA lithium diisopropylamide
Lit. literature (reference)
min minute(s)
MS mass spectroscopy
NMR nuclear magnetic resonance spectroscopy
RP-HPLC reversed phase HPLC
RT room temperature
Rₜ retention time (in HPLC)
THF tetrahydrofuran
dil. dilute
aq. aqueous

HPLC- and LC-MS methods:

Method 1 (HPLC):

Instrument: Hewlett Packard Series 1050; column: Symmetry TM C18 3.9×150 mm; flow rate: 1.5 ml/min; mobile phase A: water, mobile phase B: acetonitrile; gradient:→0.6 min 10% B→3.8 min 100% B→5.0 min 100% B→5.5 min 10% B; stop time: 6.0 min; injection volume: 10 μl; diode array detector signal: 214 and 254 nm.

Method 2 (LC-MS):

Instrument: Micromass Quattro LCZ with HPLC Agilent Series 1100; column: Phenomenex Synergi 2μ Hydro-RP Mercury 20 mm×4 mm; mobile phase A: 1 l of water+0.5 ml of 50% strength formic acid, mobile phase B: 1 l of acetonitrile+0.5 ml of 50% strength formic acid; gradient: 0.0 min 90% A→2.5 min 30% A→3.0 min 5% A→4.5 min 5% A; flow rate: 0.0 min 1 m/min→2.5 min/3.0 min/4.5 min 2 ml/min; oven: 50° C.; UV detection: 208-400 nm.

Method 3 (LC-MS):

MS instrument type: Micromass ZQ; HPLC instrument type: Waters Alliance 2795; column: Phenomenex Synergi 2μ Hydro-RP Mercury 20 mm×4 mm; mobile phase A: 1 l of water+0.5 ml of 50% strength formic acid, mobile phase B: 1 l of acetonitrile+0.5 ml of 50% strength formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 210 nm.

Method 4 (LC-MS):

MS instrument type: Micromass ZQ; HPLC instrument type: HP 1100 Series; UV DAD; column: Phenomenex Synergi 2μ Hydro-RP Mercury 20 mm×4 mm; mobile phase A: 1 l of water+0.5 ml of 50% strength formic acid, mobile phase B: 1 l of acetonitrile+0.5 ml of 50% strength formic acid; gradient: 0.0 min 90% A→2.5 min 30% A→3.0 min 5% A→4.5 min 5% A; flow rate: 0.0 min 1 ml/min→2.5 min/3.0 min/4.5 min 2 ml/min; oven: 50° C.; UV detection: 210 nm.

Method 5 (LC-MS):

MS instrument type: Micromass ZQ; HPLC instrument type: Waters Alliance 2795; column: Merck Chromolith SpeedROD RP-18e 100 mm×4.6 mm; mobile phase A: water+500 μl of 50% strength formic acid/l, mobile phase B: acetonitrile+500 μl of 50% strength formic acid/l; gradient: 0.0 min 10% B→7.0 min 95% B→9.0 min 95% B; oven: 35° C.; flow rate: 0.0 min 1.0 ml/min→7.0 min 2.0 m/min→9.0 min 2.0 ml/min; UV detection: 210 nm.

Method 6 (HPLC):

Instrument: HP 1100 with DAD detection; column: Kromasil RP-18, 60 mm×2 mm, 3.5 μm; mobile phase A: 5 ml of HClO₄/l of water, mobile phase B: acetonitrile; gradient: 0 min 2% B→0.5 min 2% B→4.5 min 90% B→9 min 90% B; flow rate: 0.75 m/min; oven: 30° C.; UV detection: 210 nm.

Method 7 (HPLC):

Instrument: HP 1100 with DAD detection; column: Kromasil RP-18, 60 mm×2 mm, 3.5 μm; mobile phase A: 5 ml of HClO₄/l of water, mobile phase B: acetonitrile; gradient: 0 min 2% B→0.5 min 2% B→4.5 min 90% B→6.5 min 90% B; flow rate: 0.75 ml/min; oven: 30° C.; UV detection: 210 nm.

Starting Materials and Intermediates:

### Example 1A

### 4-[(2,2-Dimethyl-1,3-dioxolan-4-yl)methoxy]benzaldehyde

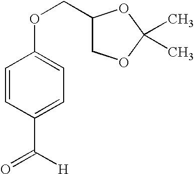

39.3 g (150 mmol) of triphenylphosphine are added to a solution of 13.2 g (100 mmol) of 1,2-O-isopropylidene glycerol in 250 ml of dry THF, and the mixture is stirred at RT for 30 min. The mixture is cooled to about 0° C., and 12.2 g (100 mmol) of 4-hydroxybenzaldehyde and 31.9 g (150 mmol) of diisopropyl azodicarboxylate (DIAD) are added. The yellow reaction solution is stirred at RT for 16 h. The mixture is then concentrated on a rotary evaporator, and the residue is added to 150 ml of sat. sodium bicarbonate solution. The mixture is extracted with ethyl acetate (three times 150 ml each), and the combined organic phases are dried over sodium sulfate. After removal of the solvent on a rotary evaporator, the crude product is purified chromatographically on silica gel 60 (mobile phase gradient cyclohexane→cyclohexane/ethyl acetate 2:1).

Yield: 5.03 g (21% of theory)

LC-MS (method 3): Rₜ=1.86 min; MS (ESIpos): m/z=237 [M+H]⁺.

### Example 2A

### {4-[(2,2-Dimethyl-1,3-dioxolan-4-yl)methoxy]benzylidene}malononitrile

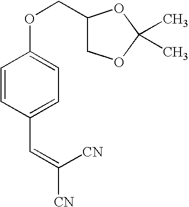

0.13 g (1.98 mmol) of malononitrile, 0.45 g (1.90 mmol) of the compound from Example 1A and 5.7 μl (0.06 mmol) of piperidine are dissolved in ethanol, and the mixture is heated under reflux for 3.5 h. The reaction solution is concentrated and the residue is purified chromatographically on silica gel 60 (mobile phase gradient cyclohexane→cyclohexane/ethyl acetate 2:1).

Yield: 0.43 g (79% of theory)

¹H-NMR (400 MHz, CDCl₃): δ=7.91 (d, 2H), 7.65 (s, 1H), 7.03 (d, 2H), 4.51 (m, 1H) 4.19 (dd, 1H), 4.14 (dd, 1H), 4.06 (dd, 1H), 3.91 (dd, 1H), 1.46 (s, 3H), 1.41 (s, 3H).

MS (DCI, NH₃): m/z=302 [M+NH₄]⁺.

### Example 3A

### 2-Amino-4-{4-[(2,2-dimethyl-1,3-dioxolan-4-yl)methoxy]phenyl}-6-mercaptopyridine-3,5-dicarbonitrile

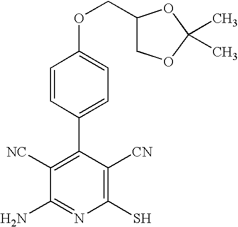

0.43 g (1.51 mmol) of the compound from Example 2A, 0.38 g (3.78 mmol) of cyanothioacetamide and 0.38 g (3.78 mmol) of 4-methylmorpholine are dissolved in 15 ml of ethanol, and the mixture is stirred under reflux for 6 h. After cooling, the reaction solution is concentrated on a rotary evaporator and the residue is chromatographed on silica gel 60. After the removal of byproducts (mobile phase gradient cyclohexane→cyclohexane/ethyl acetate 1:1), the product fractions are eluted (mobile phase gradient ethyl acetate→ethyl acetate/methanol 20:1). This is followed by fine purification via preparative HPLC (column: YMC GEL ODS-AQ S-5/15 μm; mobile phase gradient: acetonitrile/water 10:90→95:5).

Yield: 88 mg (15% of theory)

¹H-NMR (400 MHz, DMSO-d₆): δ=12.96 (br. s, 1H), 7.90 (br. s, 2H), 7.46 (d, 2H), 7.12 (d, 2H), 4.44 (m, 1H), 4.18-4.02 (m, 3H), 3.79 (m, 1H), 1.37 (s, 3H), 1.32 (s, 3H).

LC-MS (method 3): Rₜ=1.76 min; MS (ESIpos): m/z=383 [M+H]⁺.

### Example 4A

### 4-[(4-{[(6-Amino-3,5-dicyano-4-{4-[(2,2-dimethyl-1,3-dioxolan-4-yl)methoxy]phenyl}pyridin-2-yl)thio]methyl}-1,3-thiazol-2-yl)amino]benzoic acid

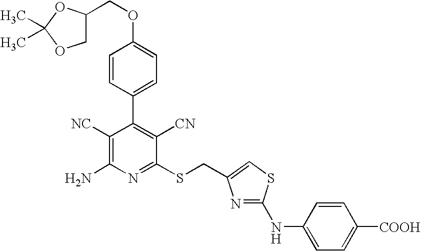

177 mg (0.90 mmol) of 4-carboxyphenylthiourea and 111 mg (0.87 mmol) of 1,3-dichloroacetone are dissolved in 3 ml of DMF, and the reaction solution is stirred at 100° C. for 60 min. After cooling, 230 mg (0.60 mmol) of the compound from Example 3A and 151 mg (1.80 mmol) of sodium bicarbonate are added, and the mixture is stirred at RT for a further 16 h. The reaction mixture is directly purified chromatographically by preparative UPLC (column: YMC GEL ODS-AQ S-5/15 μm; mobile phase gradient: acetonitrile/water 10:9043 95:5).

Yield: 134 mg (36% of theory)

¹H-NMR (400 MHz, CDCl₃): δ=12.5 (m, 1H), 10.6 (s, 1H), 8.07 (br. s, 2H), 7.86 (d, 2H), 7.67 (d, 2H), 7.49 (d, 2H), 7.12 (d, 2H), 7.07 (s, 1H), 4.50 (s, 2H), 4.44 (m, 1H), 4.16-4.03 (m, 3H), 3.78 (dd, 1H), 1.37 (s, 3H), 1.31 (s, 3H).

LC-MS (method 4): R_{t=2.51} min; MS (ESIpos): m/z=615 [M+H]⁺.

### Example 5A

### 2-Amino-4-{4-[(2,2-dimethyl-1,3-dioxolan-4-yl)methoxy]phenyl}-6-[({2-[(4-fluorophenyl)amino]-1,3-thiazol-4-yl}methyl)thio]pyridine-3,5-dicarbonitrile

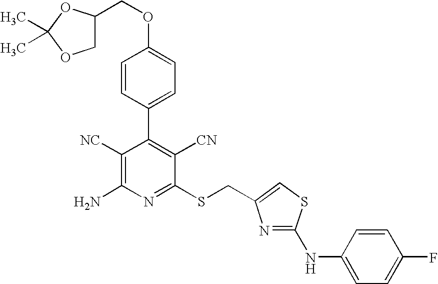

102 mg (0.6 mmol) of 4-fluorophenylthiourea and 73.6 mg (0.58 mmol) of 1,3-dichloroacetone are dissolved in 2.5 ml of ethanol, and the mixture is stirred under reflux for 60 min. The mixture is allowed to cool and concentrated on a rotary evaporator. The residue is taken up in 1.5 ml of DMF, 153 mg (0.4 mmol) of the compound from Example 3A and 101 mg (1.2 mmol) of sodium bicarbonate are added and the reaction solution is stirred at RT for a further 16 h. The reaction mixture is directly purified chromatographically by preparative HPLC (column: YMC GEL ODS-AQ S-5/15 μm; mobile phase gradient: acetonitrile/water 10:90→95:5).

Yield: 62 mg (26% of theory)

¹H-NMR (400 MHz, DMSO-d₆): δ=10.24 (s, 1H), 8.08 (br. s, 2H), 7.62 (dd, 2H), 7.47 (d, 2H), 7.13 (m, 4H), 6.97 (s, 1H), 4.49-4.39 (m, 3H), 4.10 (m, 3H), 3.78 (dd, 1H), 1.36 (s, 3H), 1.31 (s, 3H).

LC-MS (method 2): Rₜ=2.51 min; MS (ESIpos): m/z=589 [M+H]⁺.

The examples listed in Table 1 are prepared analogously to Example 5A from the appropriate starting materials:

TABLE 1

| | | MS | LC-MS | |
|---|---|---|---|---|
| Example | | (ESI) | Rₜ [min] | Yield |
| No. | Structure | [M + H]⁺ | (method) | (% of theory) |
| 6A | 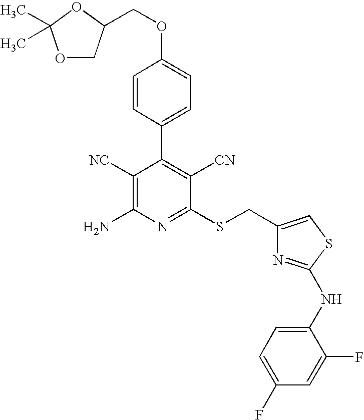 | 607 | 2.62 (3) | 39 |
| 7A | 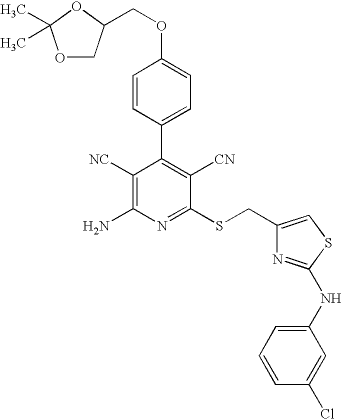 | 605 | 2.96 (4) | 53 |
| 8A | 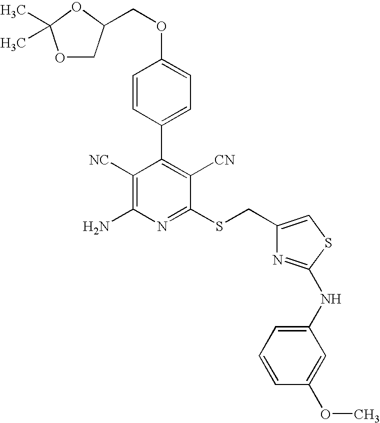 | 601 | 2.80 (4) | 40 |
| 9A | 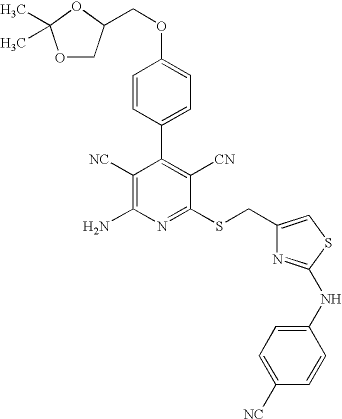 | 596 | 2.74 (4) | 38 |
| 10A | 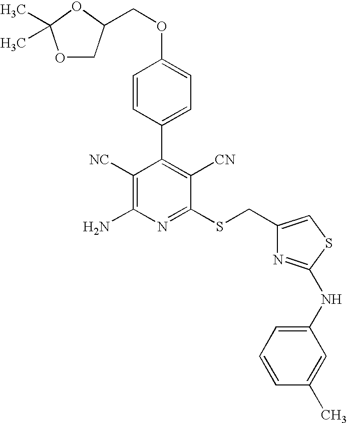 | 585 | 2.89 (4) | 46 |

### Example 11A

### (S)-4-[(2,2-Dimethyl-1,3-dioxolan-4-yl)methoxy]benzaldehyde

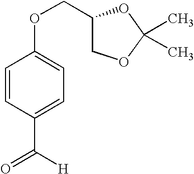

1.79 g (14.6 mmol) of p-hydroxybenzaldehyde are dissolved in absolute DMF (10 ml), and 14.2 g (102.5 mmol) of potassium carbonate and 3.3 g (22.0 mmol) of (R)-(+)-4-chloromethyl-2,2-dimethyl-1,3-dioxolane are added. The mixture is heated at 150° C. for 24 h. The mixture is then concentrated on a rotary evaporator, and the residue is partitioned between dichloromethane and water. The aqueous phase is extracted with dichloromethane (three times 20 ml each) and the combined organic phases are washed with sat. sodium chloride solution and dried over magnesium sulfate. After removal of the solvent on a rotary evaporator, the crude product is purified chromatographically on silica gel 60 (mobile phase: cyclohexane/ethyl acetate 5:1).

Yield: 2.12 g (61% of theory)

LC-MS (method 2): Rₜ=1.97 min; MS (ESIpos): m/z=237 [M+H]⁺.

### Example 12A

### (S)-2-Amino-4-{4-[(2,2-dimethyl-1,3-dioxolan-4-yl)methoxy]phenyl}-6-mercaptopyridine-3,5-dicarbonitrile

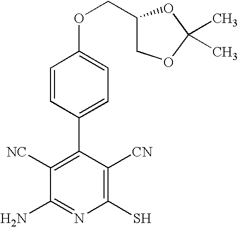

1.52 g (6.43 mmol) of the compound from Example 11A, 1.29 g (12.9 mmol) of cyanothioacetamide and 1.3 g (12.9 mmol) of 4-methylmorpholine are dissolved in 15 ml of ethanol, and the mixture is stirred under reflux for 3 h. The mixture is then stirred at RT for 18 h. The reaction solution is concentrated on a rotary evaporator, and the residue is chromatographed on silica gel 60 (mobile phase: dichloromethane/ethanol 10:1).

Yield: 1.06 g (43% of theory)

LC-MS (method 3): Rₜ=1.75 min; MS (ESIpos): m/z=383 [M+H]⁺.

### Example 13A

### (S)-2-Amino-4-{4-[(2,2-dimethyl-1,3-dioxolan-4-yl)methoxy]phenyl}-6-[({2-[(4-fluorophenyl)-amino]-1,3-thiazol-4-yl}methyl)thio]pyridine-3,5-dicarbonitrile

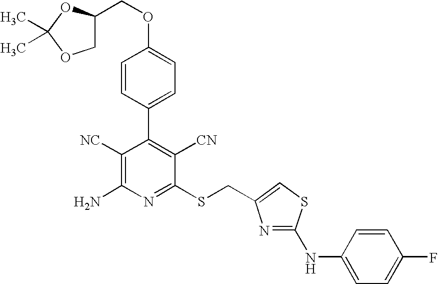

The synthesis is carried out analogously to Example 5A using enantiomerically pure starting material from Example 12A.

Yield: 47% of theory

LC-MS (method 3): Rₜ=2.58 min; MS (ESIpos): m/z=589 [M+H]⁺.

The examples listed in Table 2 are prepared from the appropriate starting materials analogously to Example 5A or 13A or the corresponding enantiomer:

TABLE 2

| | | MS | LC-MS | |
|---|---|---|---|---|
| Example | | (ESI) | Rₜ [min] | Yield |
| No. | Structure | [M + H]⁺ | (method) | (% of theory) |
| 14A | 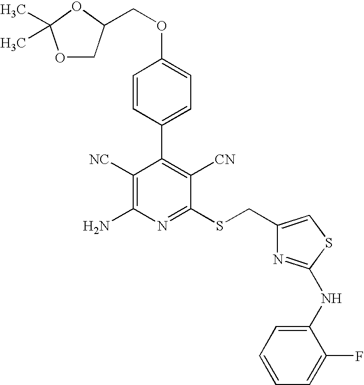 | 589 | 2.77 (2) | 50 |
| 15A | 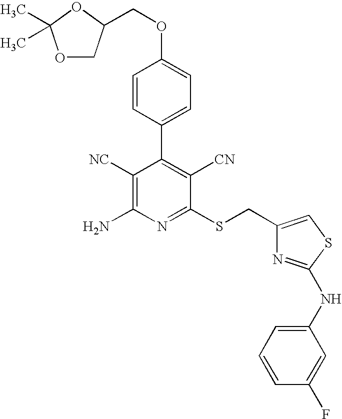 | 589 | 2.60 (3) | 36 |
| 16A | 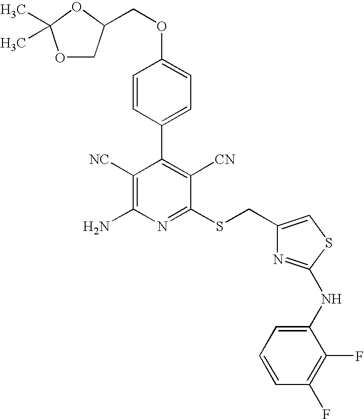 | 607 | 2.64 (3) | 52 |
| 17A | 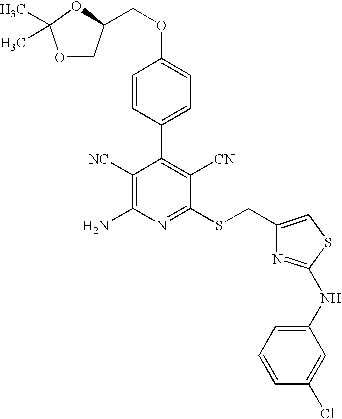 | 605 | 2.71 (3) | 33 |
| 18A | 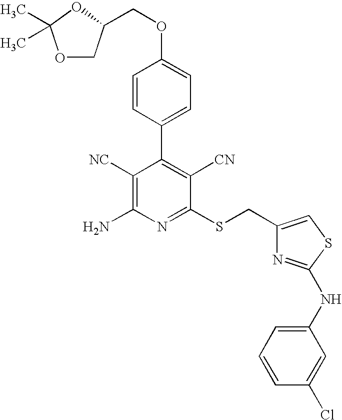 | 605 | 2.71 (3) | 54 |
| 19A | 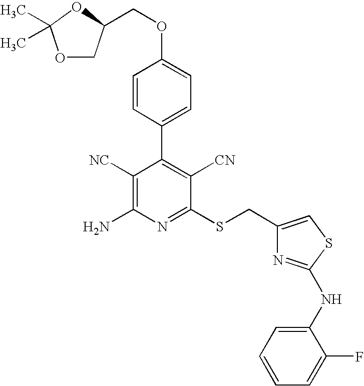 | 589 | 2.76 (2) | 61 |
| 20A | 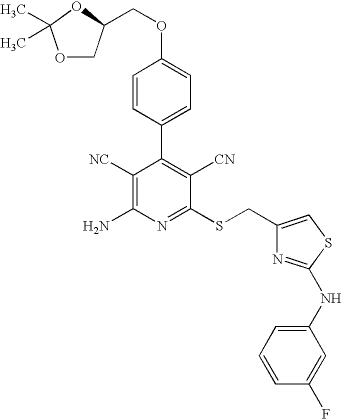 | 589 | 2.60 (3) | 18 |
| 21A | 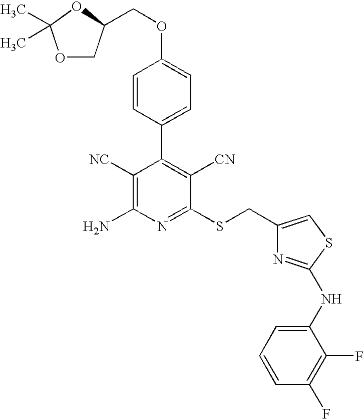 | 607 | 2.87 (4) | 41 |
| 22A | 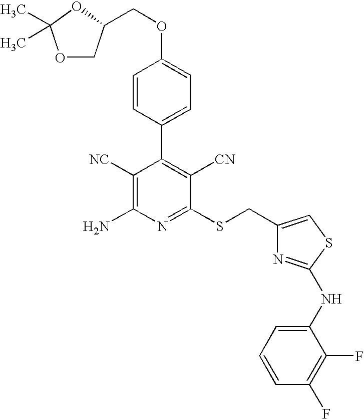 | 607 | 2.83 (2) | 23 |
| 23A | 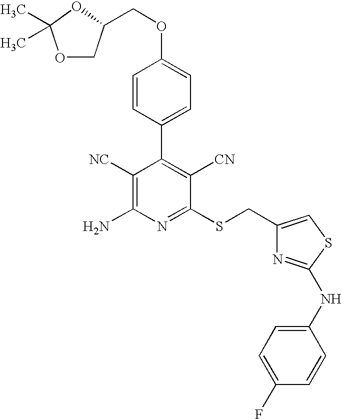 | 589 | 2.58 (3) | 80 |
| 24A | 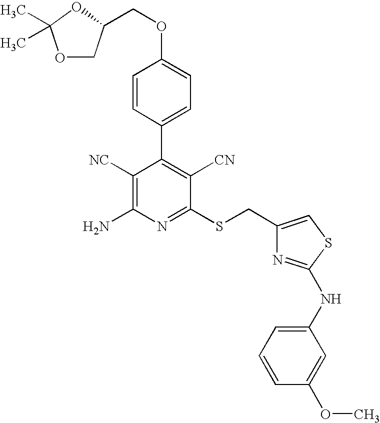 | 601 | 2.88 (4) | 67 |
| 25A | 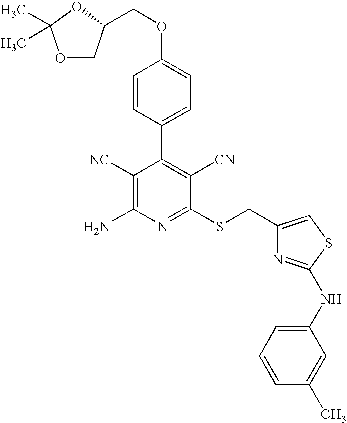 | 585 | 3.01 (4) | 62 |
| 26A | 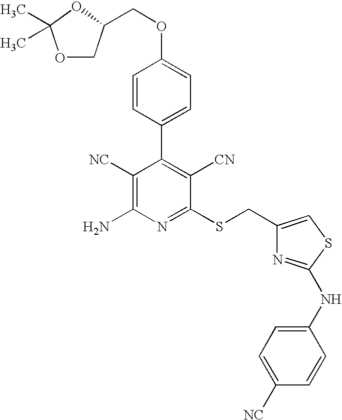 | 596 | 2.84 (4) | 45 |
| 27A | 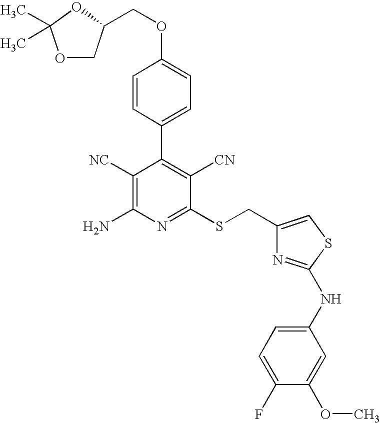 | 619 | 2.79 (4) | 68 |
| 28A | 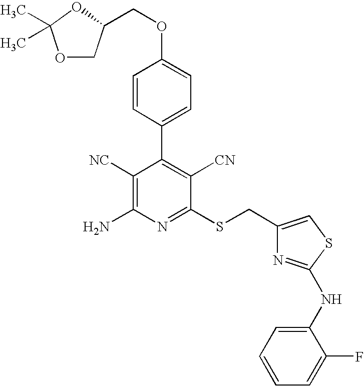 | 589 | 2.80 (4) | 66 |

### Example 29A

### 2-Amino-4-[4-(2-hydroxyethoxy)phenyl]-6-phenylthiopyridine-3,5-dicarbonitrile

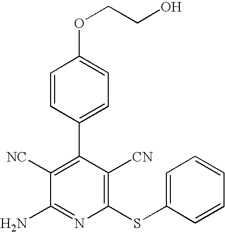

0.765 g (11.6 mmol) of malononitrile, 1.28 g (11.6 mmol) of thiophenol and 2.48 g (11.6 mmol) of 2-[4-(2-hydroxyethoxy)benzylidene]malononitrile [preparation according to WO 03/053441, Example 6/method 2, 1st. step] are dissolved in 15 ml of ethanol, and 0.03 ml of triethylamine is added. The mixture is stirred under reflux for 2 h. After cooling, the reaction mixture is filtered and the residue is washed with ethanol and dried.

Yield: 2.07 g (46% of theory)

¹H-NMR (400 MHz, DMSO-d₆): δ=7.76 (br. s, 2H), 7.60 (m, 2H), 7.51 (m, 5H), 7.12 (d, 2H), 4.93 (t, 1H), 4.09 (t, 2H), 3.75 (m, 2H).

LC-MS (method 3): Rₜ=2.02 min; MS (ESIpos): m/z=389 [M+H]⁺.

### Example 30A

### 2-Chloro-4-[4-(2-hydroxyethoxy)phenyl]-6-phenylthiopyridine-3,5-dicarbonitrile

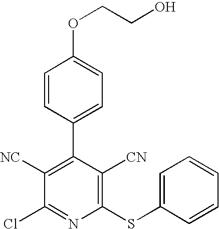

2.07 g (5.33 mmol) of the compound from Example 29A are dissolved in 11 ml of absolute DMF, and 4.30 g (32.0 mmol) of anhydrous copper(II) chloride and 2.71 ml (32.0 mmol) of isoamyl nitrite are added. The mixture is stirred at 40° C. for 18 h. The reaction solution is then concentrated on a rotary evaporator, and the residue is added to 1 N hydrochloric acid. The mixture is extracted three times with dichloromethane, and the combined organic phases are washed with 1 N hydrochloric acid and sodium chloride solution. After drying over magnesium sulfate, the solvent is removed on a rotary evaporator. The crude product is purified chromatographically on silica gel 60 (mobile phase: dichloromethane/ethanol 20:1).

Yield: 1.29 g (59% of theory)

¹H-NMR (400 MHz, DMSO-d₆): δ=7.60 (m, 7H), 7.20 (d, 2H), 4.12 (t, 2H), 3.76 (t, 2H).

LC-MS (method 3): Rₜ=2.38 min; MS (ESIpos): m/z=408 [M+H]⁺.

### Example 31A

### 2-(2-Hydroxyethoxy)amino-4-[4-(2-hydroxyethoxy)phenyl]-6-phenylthiopyridine-3,5-dicarbonitrile

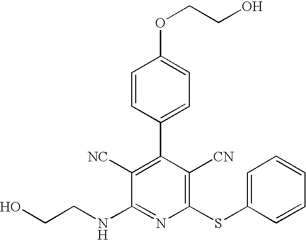

0.50 g (1.23 mmol) of the compound from Example 30A is dissolved in 1.5 ml of DMF, and 0.16 ml (2.70 mmol) of 2-hydroxyethylamine is added. The mixture is allowed to stir for 20 min, and 2 ml of methanol and 4 ml of water are then added. The precipitate is filtered off, washed with methanol and dried.

Yield: 0.36 g (68% of theory)

¹H-NMR (400 MHz, DMSO-d₆): δ=7.94 (br. s, 1H), 7.55 (m, 7H), 7.13 (d, 2H), 4.93 (t, 1H), 4.49 (t, 1H), 4.09 (t, 2H), 3.75 (m, 2H), 3.09 (m, 2H), 3.00 (m, 2H).

LC-MS (method 4): Rₜ=2.33 min; MS (ESIpos): m/z=433 [M+H]⁺.

### Example 32A

### 2-(2-Hydroxyethoxy)amino-4-[4-(2-hydroxyethoxy)phenyl]-6-mercaptopyridine-3,5-dicarbonitrile

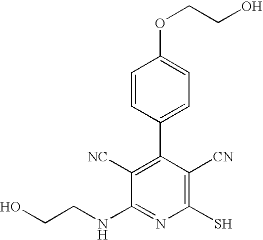

0.30 g (0.70 mmol) of the compound from Example 31A are dissolved in 2 ml of DMF, and 0.19 g (2.43 mmol) of sodium sulfide are added. The mixture is stirred at 80° C. for 2 h and then at RT for 12 h. 1 N hydrochloric acid (10 ml) is then added, and the precipitate is filtered off.

Yield: 0.25 g (72% of theory)

LC-MS (method 3): Rₜ=1.21 min; MS (ESIpos): m/z=357 [M+H]⁺.

### Example 33A

### 2-Amino-6-(benzylthio)-4-{4-[2-(dimethylamino)ethoxy]phenyl}pyridine-3,5-dicarbonitrile

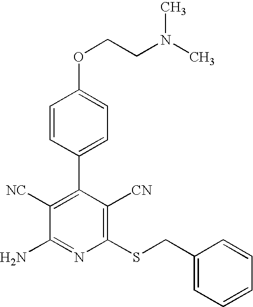

6.31 g (56.2 mmol) of potassium tert-butoxide are added to a solution of 8.39 g (23.4 mmol) of 2-amino-6-(benzylthio)-4-(4-hydroxyphenyl)pyridine-3,5-dicarbonitrile [preparation according to WO 01/25210, Example A 383, from 2-amino-4-(4-hydroxyphenyl)-6-mercaptopyridine-3,5-dicarbonitrile and benzyl bromide] in 105.5 ml of ethanol. The mixture is stirred at RT for 1 h, and 4.05 g (28.1 mmol) of 2-dimethylaminoethyl chloride hydrochloride are then added. The mixture is then stirred at +78° C. for 3 h. After cooling, the reaction mixture is filtered and the filtrate is concentrated on a rotary evaporator. The residue is purified directly by preparative HPLC (column: Merck 210 g RP-silica gel Gromsil 120 ODS-4 HR 10 μm, 50 mm×200 mm; mobile phase A=water+0.1% formic acid, mobile phase B=acetonitrile; gradient: 0 min 10% B→5 min 10% B→6 min 90% B→22 min 90% B→22 min 10% B→28 min 10% B; flow rate: 110 ml/min; wavelength: 220 nm).

Yield: 3.55 g (35% of theory)

LC-MS (method 3): Rₜ=1.57 min; MS (ESIpos): m/z=430 [M+H]⁺.

### Example 34A

### 2-Amino-4-{4-[2-(dimethylamino)ethoxy]phenyl}-6-mercaptopyridine-3,5-dicarbonitrile

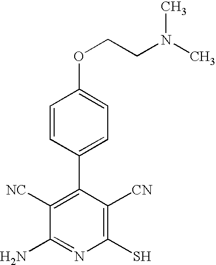

0.97 g (12.41 mmol) of sodium sulfide is added to a solution of 3.56 g (8.28 mmol) of the compound from Example 33A in 13 ml of dry DMF. The reaction mixture is stirred at +80° C. for 2 h. After cooling to RT, 2 ml of 37% strength hydrochloric acid are added to the reaction mixture, resulting in the mixture warming to 65° C. After addition of 2.6 ml of water, the reaction mixture is cooled back to RT. After addition of a further 5 ml of water, the mixture is washed with 5 ml of ethyl acetate and made alkaline by addition of 40% strength aqueous sodium hydroxide solution. Yellow crystals precipitate out, which are filtered off with suction and washed with 10 ml of water and 10 ml of diethyl ether and then dried under reduced pressure. The filtrate is concentrated on a rotary evaporator and triturated with a little water. The crystals obtained are filtered off with suction, washed with 10 ml each of water and diethyl ether and dried under reduced pressure.

Yield: 0.38 g (13% of theory)

¹H-NMR (400 MHz, DMSO-d₆): δ=10.77 (br. s, 1H), 7.39 (d, 2H), 7.09 (d, 2H), 6.92 (br. s, 2H), 4.30 (t, 2H), 3.21 (br. s, 2H), 2.64 (s, 6H).

LC-MS (method 3): Rₜ=0.83 min; MS (ESIpos): m/z=340 [M+H]⁺.

### Example 35A

### 2-(4-Formylphenoxy)ethyl 4-methylphenylsulfonate

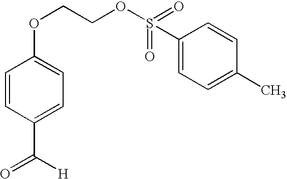

At RT, 12.6 ml (90.3 mmol) of triethylamine and a solution of 13.77 g (72.2 mmol) of toluene-4-sulfonyl chloride in 200 ml of dichloromethane are successively added dropwise with stirring to a solution of 10.0 g (60.2 mmol) of 4-(2-hydroxyethoxy)benzaldehyde in 300 ml of dichloromethane. The reaction mixture is stirred at RT for 12 h. After addition of 0.15 g (1.2 mmol) of 4-N,N-dimethylaminopyridine, the mixture is stirred at RT for a further 4 h. 250 ml of saturated aqueous sodium bicarbonate solution are then added, and the mixture is extracted three times with in each case 100 ml of dichloromethane. The combined organic phases are dried over sodium sulfate. After removal of the solvent on a rotary evaporator, the crude product is purified chromatographically on silica gel 60 (mobile phase gradient cyclohexane→ethyl acetate).

Yield: 12.34 g (64% of theory)

HPLC (method 6): Rₜ=4.57 min; MS (ESIpos): m/z=321 [M+H]⁺.

### Example 36A

### 4-(2-Azidoethoxy)benzaldehyde

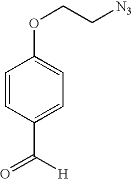

A solution of 5.0 g (15.61 mmol) of the compound from Example 35A and 2.03 g (31.22 mmol) of sodium azide in 100 ml of dry DMF is stirred at RT for 12 h. The reaction mixture is concentrated on a rotary evaporator, and the residue is suspended in about 20 ml of water. After three extractions with in each case 30 ml of diethyl ether, the combined organic phases are washed twice with in each case 10 ml of water and once with 10 ml of saturated sodium chloride solution. After drying over sodium sulfate, the solvent is removed on a rotary evaporator.

Yield: 3.02 g (98% of theory)

HPLC (method 7): Rₜ=4.14 min; MS (DCI): m/z=209 [M+NH₄]⁺.

### Example 37A

### [4-(2-Azidoethoxy)benzylidene]malononitrile

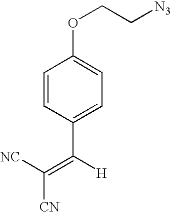

47 μl (0.47 mmol) of piperidine are added dropwise to a solution of 3.02 g (15.79 mmol) of the compound from Example 36A and 1.09 g (16.42 mmol) of malononitrile in 100 ml of ethanol, and the reaction mixture is stirred at +78° C. for 3.5 h. The colour of the solution changes to orange-red. After cooling to RT, the solution is allowed to stand without stirring for 20 h. A colourless precipitate is formed. On a rotary evaporator, the crude suspension is concentrated to half of the original volume, and crystallization is completed with cooling in an ice-bath. The precipitate obtained is filtered off with suction and washed twice with in each case 20 ml of ethanol and twice with in each case 20 ml of methyl tert-butyl ether.

Yield: 2.38 g (63% of theory)

MS (DCI): m/z=257 [M+NH₄]⁺.

### Example 38A

### 2-Amino-4-[4-(2-azidoethoxy)phenyl]-6-mercaptopyridine-3,5-dicarbonitrile

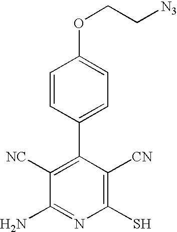

A solution of 2.39 g (9.98 mmol) of the compound from Example 37A and 2.50 g (24.92 mmol) of cyanothioacetamide in 100 ml of ethanol is stirred at +78° C. for 6 h. After cooling to RT and 12 h of standing without stirring, the reaction mixture is concentrated on a rotary evaporator. The residue is recrystallized from about 30 ml of ethanol. The precipitate obtained is filtered off with suction and washed twice with in each case 10 ml of methyl tert-butyl ether.

Yield: 2.04 g (61% of theory)

MS (DCI): m/z=355 [M+NH]⁺.

### Example 39A

### 2-Amino-4-[4-(2-azidoethoxy)phenyl]-6-[({2-[(4-fluorophenyl)amino]-1,3-thiazol-4-yl}methyl)-thio]pyridine-3,5-dicarbonitrile

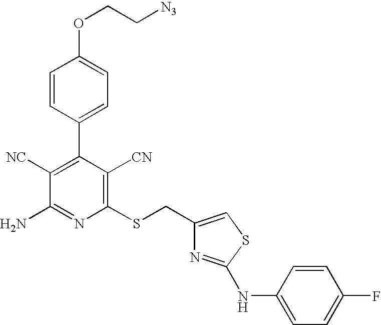

A solution of 74 mg (0.44 mmol) 4-fluorophenylthiourea and 55 mg (0.44 mmol) 1,3-dichloro-acetone in 5 ml ethanol is stirred at +85° C. for 60 min. After removal of the solvent on a rotary evaporator, the residue is taken up in 5 ml of DMF, 105 mg (0.31 mmol) of the compound from example 38A and 78 mg (0.93 mmol) of sodium bicarbonate are added and the mixture is then stirred at RT for 20 h. The mixture is then added to 15 ml of saturated sodium bicarbonate solution. The mixture is extracted with ethyl acetate (three times 15 ml each), and the combined organic phases are dried over magnesium sulfate. After removal of the solvent on a rotary evaporator, the crude product is purified chromatographically on silica gel 60 (mobile phase gradient dichloromethane/ethanol 200:1→20:1).

Yield: 79 mg (47% of theory)

¹H-NMR (400 MHz, DMSO-d₆): δ=10.22 (s, 1H), 8.27-7.86 (br. s, 2H), 7.66-7.58 (m, 2H), 7.50 d, 2H), 7.17-7.08 (m, 4H), 6.96 (s, 1H), 4.46 (s, 2H), 4.31-4.22 (m, 2H), 3.74-3.67 (m, 2H).

LC-MS (method 2): Rₜ=2.72 min; MS (ESIpos): m/z=544 [M+H]⁺.

### Example 40A

### 4-(2-Hydroxypropoxy)benzaldehyde

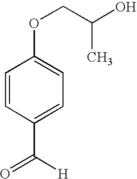

18.30 g (172.6 mmol) of sodium carbonate are added to a solution of 7.03 g (57.5 mmol) of p-hydroxybenzaldehyde and 6.80 g (71.9 mmol) of 1-chloro-2-propanol (technical grade, about 70%, isomer mixture with 2-chloro-1-propanol) in 125 ml of dry DMF, and the mixture is stirred at +130° C. for 20 h. After cooling to RT, 100 ml of saturated sodium bicarbonate solution are added, and the mixture is extracted with ethyl acetate (three times 100 ml each). The combined organic phases are dried over magnesium sulfate. After removal of the solvent on a rotary evaporator, the crude product is purified chromatographically on silica gel 60 (mobile phase gradient cyclohexane/ethyl acetate 5:1→2:1).

Yield: 4.60 g (44% of theory, isomer mixture, ratio 75:25)

LC-MS (method 4): Rₜ=1.63 min; MS (ESIpos): m/z=181 [M+H]⁺.

### Example 41A

### 4-(2-{[tert-Butyl(dimethyl)silyl]oxy}propoxy)benzaldehyde

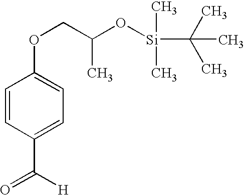

5.39 g (35.7 mmol) of tert-butyldimethylsilyl chloride and 3.30 g (48.5 mmol) of imidazole are added successively to a solution of 4.60 g (25.5 mmol) of the compound from example 40A in 120 ml of dry dimethylformamide, and the mixture is stirred at RT for 20 h. 100 ml of saturated sodium bicarbonate solution are then added, and the reaction mixture is extracted with diethyl ether (three times 100 ml each). The combined organic phases are dried over magnesium sulfate. After removal of the solvent on a rotary evaporator, the crude product is purified chromatographically on silica gel 60 (mobile phase gradient cyclohexane/ethyl acetate 50:1→10:1).

Yield: 4.00 g (53% of theory, isomer mixture, ratio 86:14)

LC-MS (method 2): Rₜ=3.29 min; MS (ESIpos): m/z=295 [M+H]⁺.

### Example 42A

### 2-Amino-4-[4-(2-{[tert-butyl(dimethyl)silyl]oxy}propoxy)phenyl]-6-mercaptopyridine-3,5-dicarbonitrile

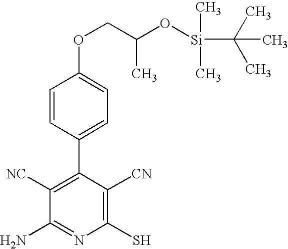

A solution of 1.77 g (5.99 mmol) of the compound from example 41A and 1.26 g (12.59 mmol) of cyanothioacetamide in 25 ml of ethanol is stirred at +78° C. for 6 h. The mixture is then cooled to RT and stirred at this temperature for a further 20 h. The resulting precipitate is filtered off with suction and washed with cold diethyl ether. Further product is obtained from the concentrated filtrate solution by chromatographic purification on silica gel 60 (Mobile phase gradient cyclohexane/ethyl acetate 1:1→1:4).

Yield: 0.25 g (9% of theory, isomer mixture)

LC-MS (method 3): Rₜ=2.71 min, 2.77 min; MS (ESIpos): m/z=430 [M+H]⁺.

### Example 43A

### 2-Amino-4-[4-(2-{[tert-butyl(dimethyl)silyl]oxy}propoxy)phenyl]-6-[({2-[(4-fluorophenyl)amino]-1,3-thiazol-4-yl}methyl)thio]pyridine-3,5-dicarbonitrile

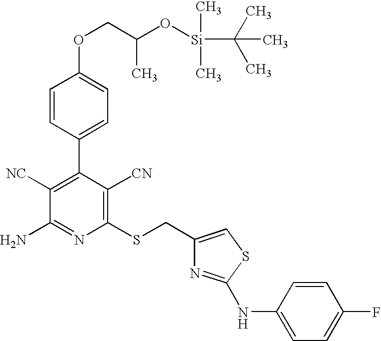

A solution of 78.6 mg (0.46 mmol) of 4-fluorophenylthiourea and 56.0 mg (0.44 mmol) of 1,3-dichloroacetone in 2 ml of dry DMF is stirred at +80° C. for 3 h. After cooling to RT, 370 mg (0.42 mmol) of the compound from example 42A are added, and the mixture is then stirred at RT for 20 h. The reaction mixture is purified directly twice by preparative HPLC (column: YMC GEL ODS-AQ S-5/15 μm; mobile phase gradient: acetonitrile/water 10:90→95:5).

Yield: 0.44 g (14% of theory)

¹H-NMR (400 MHz, DMSO-d₆): δ=10.21 (s, 1H), 8.18-7.93 (br. s, 2H), 7.60 (dd, 2H), 7.47 (d, 2H), 7.12 (t, 2H), 7.07 (d, 2H), 6.95 (s, 1H), 4.44 (s, 2H), 4.21-4.12 (m, 1H), 3.96 (dd, 1H), 3.87 (dd, 1H), 1.18 (d, 3H), 0.87 (s, 9H), 0.08 (d, 6H).

LC-MS (method 5): Rₜ=7.35 min; MS (ESIpos): m/z=647 [M+H]⁺.

### Working Examples

### Example 1

### 2-Amino-6-[({2-[(3-chlorophenyl)amino]-1,3-thiazol-4-yl}methyl)thio]-4-[4-(2,3-dihydroxypropoxy)phenyl]pyridine-3,5-dicarbonitrile

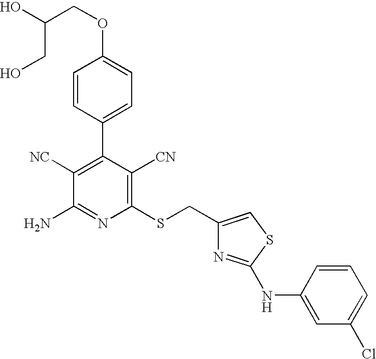

2 ml of glacial acetic acid and 90 mg (0.15 mmol) of the compound from example 7A are added to 1 ml of water, and the mixture is stirred at RT for 16 h. The mixture is concentrated and the residue is chromatographed on silica gel 60 (mobile phase gradient dichloromethane→dichloromethane/methanol 10:1).

Yield: 30 mg (36% of theory)

M.p.: 192-194° C.

¹H-NMR (400 MHz, DMSO-d₆): δ=10.42 (s, 1H), 8.06 (br. s, 2H), 7.82 (s, 1H), 7.45 (m, 3H), 7.30 (t, 1H), 7.10 (d, 2H), 7.03 (s, 1H), 6.97 (d, 1H), 4.99 (d, 1H), 4.68 (dd, 1H), 4.49 (s, 2H), 4.09 (dd, 1H), 3.95 (dd, 1H), 3.82 (m, 1H), 3.46 (dd, 2H).

LC-MS (method 3): Rₜ=2.17 min; MS (ESIpos): m/z=565 [M+H]⁺.

The examples listed in table 3 are prepared analogously to example 1 from the appropriate starting materials:

TABLE 3

| | | MS | LC-MS | | Yield |
|---|---|---|---|---|---|
| | | (ESI) | Rₜ [min] | | (% of |
| Example No. | Structure | [M + H]⁺ | (method) | ¹H-NMR | theory) |
| 2 | 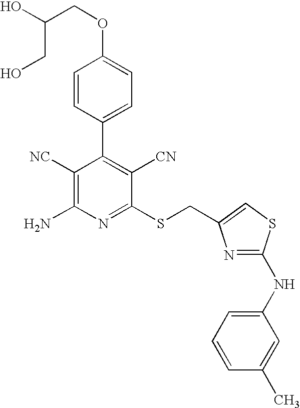 | 545 | 2.29 (2) | | 77 |
| 3 | 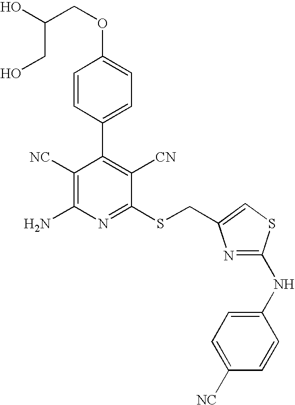 | 556 | 2.16 (2) | | 63 |
| 4 | 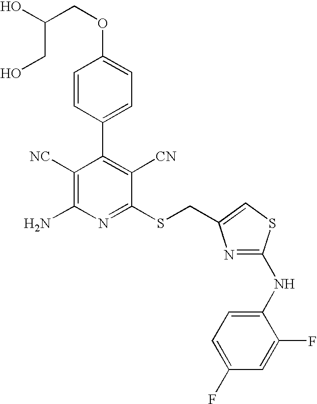 | 567 | 2.26 (2) | | 98 |
| 5 | 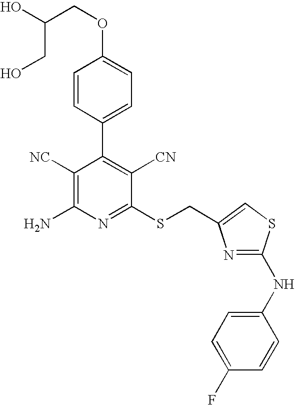 | 549 | 2.30 (4) | δ (400 MHz, DMSO- d₆) = 10.24(s, 1H), 8.09(br. s, 2H), 7.61 (dd, 2H), 7.46(d, 2H), 7.18-7.05(m, 4H), 6.97(s, 1H), 5.00(d, 1H), 4.70 (dd, 1H), 4.45(s, 2 H), 4.09(dd, 1H), 3.95(dd, 1H), 3.81 (m, 1H), 3.46(dd, 2H). | 83 |
| 6 | 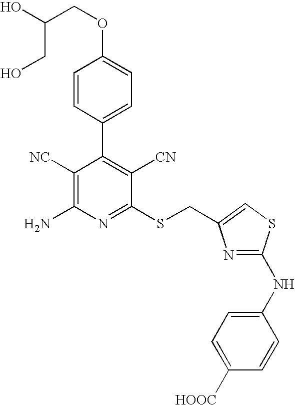 | 575 | 2.02 (4) | δ (400 MHz, DMSO- d₆) = 10.63(s, 1H), 8.10(br. s, 2H), 7.86 (d, 2H), 7.67(d, 2H), 7.49(d, 2H), 7.10(d, 2H), 7.08(s, 1H), 5.01(br. s, 1H), 4.71 (br. m, 1H), 4.09(dd, 1H), 3.95(dd, 1H), 3.81(m, 1H), 3.46(d, 1 H), 3.04(m, 1H). | 67 |
| 7 | 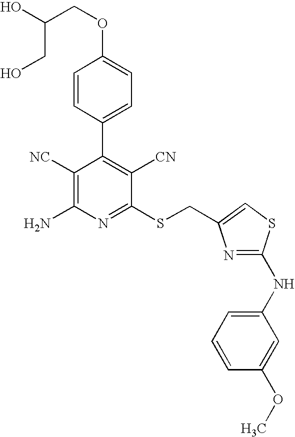 | 561 | 2.28 (4) | | 87 |

### Example 8

### 2-Amino-6-[({2-[(4-fluorophenyl)amino]-1,3-thiazol-4-yl}methyl)thio]-4-[4-(2-hydroxyethoxy)-phenyl]pyridine-3,5-dicarbonitrile

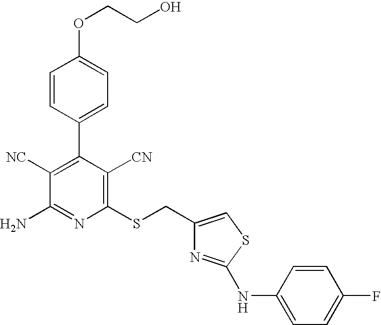

244 mg (1.92 mmol) of 1,3-dichloroacetone are added to a solution of 327 mg (1.92 mmol) of 4-fluorophenylthiourea in 8 ml of ethanol, and the mixture is stirred under reflux for 1 h. The mixture is concentrated, the residue is dissolved in 4 ml of DMF, 429 mg (1.37 mmol) of 2-amino-4-[4-(2-hydroxyethoxy)phenyl]-6-mercaptopyridine-3,5-dicarbonitrile (preparation see WO 03/053441, example 6/method 1, 1st step) and 346 mg (4.1 mmol) of sodium bicarbonate are added and the mixture is stirred at RT overnight. After addition of water, the precipitate is decanted off and the residue is triturated with dichloromethane. Chromatography on silica gel (mobile phase dichloromethane/methanol 50:1) gives the title compound as a yellowish solid.

Yield: 316 mg (44% of theory)

HPLC (method 1): Rₜ=4.24 min

¹H-NMR (400 MHz, DMSO-d₆): δ=10.23 (s, 1H), 8.1 (br. s, 2H), 7.62 (dd, 2H), 7.47 (d, 2H), 7.12 (dd, 4H), 6.96 (s, 1H), 4.92 (t, 1H), 4.45 (s, 2H), 4.07 (t, 2H), 3.74 (q, 2H).

LC-MS (method 2): Rₜ=2.39 min; MS (ESIpos): m/z=519 [M+H]⁺.

### Example 9

### 2-Amino-6-[({2-[(4-chlorophenyl)amino]-1,3-thiazol-4-yl}methyl)thio]-4-[4-(2-hydroxyethoxy)-phenyl]pyridine-3,5-dicarbonitrile

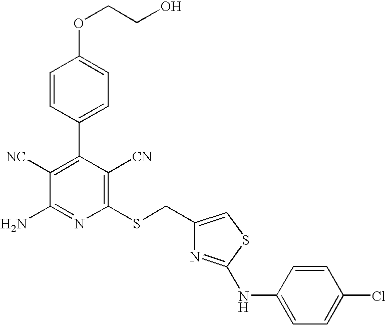

Analogously to example 8, the title compound is obtained by reacting 179 mg (0.96 mmol) of 4-chlorophenylthiourea with 122 mg (0.96 mmol) of 1,3-dichloroacetone in ethanol, followed by reaction with 150 mg (0.48 mmol) of 2-amino-4-[4-(2-hydroxyethoxy)phenyl]-6-mercaptopyridine-3,5-dicarbonitrile.

Yield: 60 mg (12% of theory)

HPLC (method 1): Rₜ=4.44 min

¹H-NMR (400 MHz, DMSO-d₆): δ=10.37 (s, 1H), 8.1 (br. s, 2H), 7.63 (d, 2H), 7.47 (d, 2H), 7.32 (d, 2H), 7.11 (d, 2H), 6.99 (s, 1H), 4.47 (s, 2H), 4.08 (t, 2H), 3.75 (q, 2H).

LC-MS (method 3): Rₜ=2.31 min; MS (ESIpos): m/z=535 [M+H]⁺.

### Example 10

### 2-Amino-6-[({2-[(2,4-difluorophenyl)amino]-1,3-thiazol-4-yl}methyl)thio]-4-[4-(2-hydroxyethoxy)phenyl]pyridine-3,5-dicarbonitrile

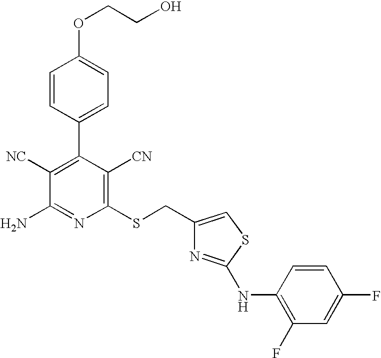

Analogously to example 8, the title compound is obtained by reacting 169 mg (0.90 mmol) of 2,4-difluorophenylthiourea with 114 mg (0.90 mmol) of 1,3-dichloroacetone in ethanol followed by reaction with 200 mg (0.64 mmol) of 2-amino-4-[4-(2-hydroxyethoxy)phenyl]-6-mercaptopyridine-3,5-dicarbonitrile.

Yield: 126 mg (36% of theory)

HPLC (method 1): Rₜ=4.31 min

¹H-NMR (400 MHz, DMSO-d₆): δ=9.97 (s, 1H), 8.34 (dt, 1H), 8.1 (br. s, 2H), 7.47 (d, 2H), 7.30 (dq, 1H), 7.10 (d, 2H), 7.04 (br. t, 1H), 6.99 (s, 1H), 4.91 (t, 1H), 4.45 (s, 2H), 4.06 (t, 2H), 3.74 (q, 2H).

LC-MS (method 3): Rₜ=2.21 min; MS (ESIpos): m/z=537 [M+H]⁺.

### Example 11

### 2-Amino-6-[({2-[(3-fluorophenyl)amino]-1,3-thiazol-4-yl}methyl)thio]-4-[4-(2-hyroxyethoxy)-phenyl]pyridine-3,5-dicarbonitrile

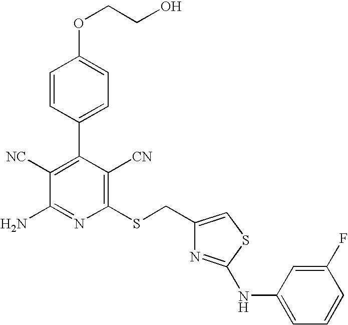

Analogously to example 8, the title compound is obtained by reacting 153 mg (0.90 mmol) of 3-fluorophenylthiourea with 114 mg (0.90 mmol) of 1,3-dichloroacetone in ethanol, followed by reaction with 200 mg (0.64 mmol) of 2-amino-4-[4-(2-hydroxyethoxy)phenyl]-6-mercaptopyridine-3,5-dicarbonitrile.

Yield: 80 mg (24% of theory)

HPLC (method 1): Rₜ=4.36 min

¹H-NMR (400 MHz, DMSO-d₆): δ=10.46 (s, 1H), 8.1 (br. s, 2H), 7.66 (dt, 1H), 7.47 (d, 2H), 7.35-7.21 (m, 2H), 7.10 (t, 2H), 7.04 (s, 1H), 6.74 (dt, 1H), 4.92 (br. s, 1H), 4.48 (s, 2H), 4.07 (t, 2H), 3.74 (t, 2H).

LC-MS (method 3): Rₜ=2.20 min; MS (ESIpos): m/z=519 [M+H]⁺.

### Example 12

### 2-Amino-6-[({2-[(2-fluorophenyl)amino]-1,3-thiazol-4-yl}methyl)thio]-4-[4-(2-hydroxyethoxy)-phenyl]pyridine-3,5-dicarbonitrile

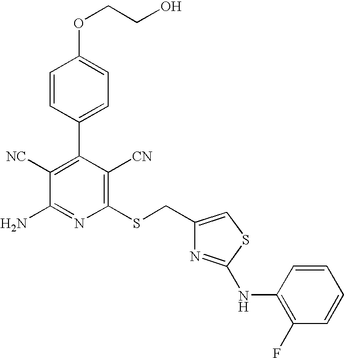

Analogously to example 8, the title compound is obtained by reacting 153 mg (0.90 mmol) of 2-fluorophenylthiourea with 114 mg (0.90 mmol) of 1,3-dichloroacetone in ethanol, followed by reaction with 200 mg (0.64 mmol) of 2-amino-4-[4-(2-hydroxyethoxy)phenyl]-6-mercaptopyridine-3,5-dicarbonitrile.

Yield: 170 mg (51% of theory)

HPLC (method 1): Rₜ=4.28 min

¹H-NMR (400 MHz, DMSO-d₆): δ=9.99 (s, 1H), 8.36 (t, 1H), 8.1 (br. s, 2H), 7.46 (d, 2H), 7.22 (dd, 1H), 7.16-7.08 (m, 3H), 7.02-6.96 (m, 2H), 4.90 (t, 1H), 4.46 (s, 2H), 4.07 (t, 2H), 3.74 (t, 2H).

LC-MS (method 3): Rₜ=2.16 min; MS (ESIpos): m/z=519 [M+H]⁺.

### Example 13

### 4-({4-[({6-Amino-3,5-dicyano-4-[4-(2-hydroxyethoxy)phenyl]pyridin-2-yl}thio)methyl]-1,3-thiazol-2-yl}amino)benzoic acid

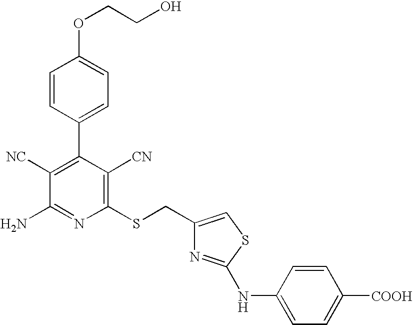

Analogously to example 8, the title compound is obtained by reacting 176 mg (0.90 mmol) of 4-[(aminocarbonothioyl)amino]benzoic acid with 114 mg (0.90 mmol) of 1,3-dichloroacetone in ethanol, followed by reaction with 200 mg (0.64 mmol) of 2-amino-4-[4-(2-hydroxyethoxy)-phenyl]-6-mercaptopyridine-3,5-dicarbonitrile.

Yield: 45 mg (13% of theory)

HPLC (method 1): Rₜ=3.81 min

¹H-NMR (300 MHz, DMSO-d₆): δ=12.6 (br. s, 1H), 10.64 (s, 1H), 8.1 (br. s, 2H), 7.87 (d, 2H), 7.68 (d, 2H), 7.49 (d, 2H), 7.13-7.06 (m, 3H), 4.45 (s, 2H), 4.07 (t, 2H), 3.74 (t, 2H).

LC-MS (method 2): Rₜ=1.97 min; MS (ESIpos): m/z=545 [M+H]⁺.

As a byproduct, this reaction yields ethyl 4-({4-[({6-amino-3,5-dicyano-4-[4-(2-hydroxyethoxy)-phenyl]pyridin-2-yl}thio)methyl]-1,3-thiazol-2-yl}amino)benzoate (see example 14).

### Example 14

### Ethyl 4-({4-[({6-amino-3,5-dicyano-4-[4-(2-hydroxyethoxy)phenyl]pyridin-2-yl}thio)methyl]-1,3-thiazol-2-yl}amino)benzoate

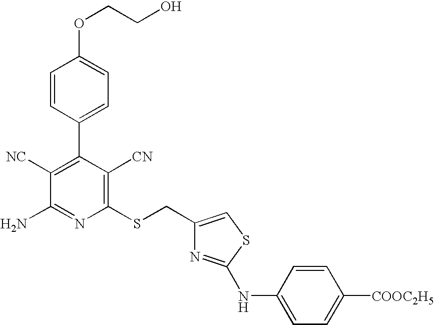

As described in example 13, the title compound is obtained as a byproduct in the reaction of 176 mg (0.90 mmol) of 4-[(aminocarbonothioyl)amino]benzoic acid with 114 mg (0.90 mmol) of 1,3-dichloroacetone in ethanol, followed by reaction with 200 mg (0.64 mmol) of 2-amino-4-[4-(2-hydroxyethoxy)phenyl]-6-mercaptopyridine-3,5-dicarbonitrile.

Yield: 59 mg (16% of theory)

HPLC (method 1): Rₜ=4.32 min

¹H-NMR (300 MHz, DMSO-d₆): δ=10.67 (s, 1H), 8.1 (br. s, 2H), 7.88 (d, 2H), 7.68 (d, 2H), 7.47 (d, 2H), 7.13-7.07 (m, 3H), 4.91 (br. s, 1H), 4.50 (s, 2H), 4.26 (q, 2H), 4.07 (t, 2H), 3.74 (t, 2H), 1.29 (t, 3H).

LC-MS (method 2): Rₜ=2.46 min; MS (ESIpos): m/z=573 [M+H]⁺.

### Example 15

### 2-Amino-4-[4-(2-hydroxyethoxy)phenyl]-6-[({2-[(4-nitrophenyl)amino]-1,3-thiazol-4-yl}methyl)-thio]pyridine-3,5-dicarbonitrile

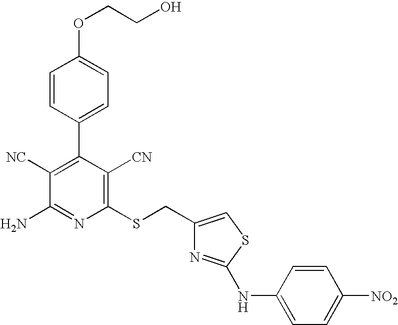

Analogously to example 8, the title compound is obtained by reacting 177 mg (0.90 mmol) of 4-nitrophenylthiourea with 114 mg (0.90 mmol) of 1,3-dichloroacetone in ethanol, followed by reaction with 200 mg (0.64 mmol) of 2-amino-4-[4-(2-hydroxyethoxy)phenyl]-6-mercaptopyridine-3,5-dicarbonitrile.

Yield: 67 mg (19% of theory)

HPLC (method 1): Rₜ=4.23 min

¹H-NMR (400 MHz, DMSO-d₆): δ=11.03 (s, 1H), 8.20 (d, 2H), 8.1 (br. s, 2H), 7.80 (d, 2H), 7.48 (d, 2H), 7.20 (s, 1H), 7.10 (d, 2H), 4.90 (t, 1H), 4.52 (s, 2H), 4.07 (t, 2H), 3.74 (q, 2H).

LC-MS (method 2): Rₜ=2.39 min; MS (ESIpos): m/z=546 [M+H]⁺.

### Example 16

### 2-Amino-4-[4-(2-hydroxyethoxy)phenyl]-6-{[(2-{[3-(trifluoromethyl)phenyl]amino}-1,3-thiazol-4-yl)methyl]thio}pyridine-3,5-dicarbonitrile

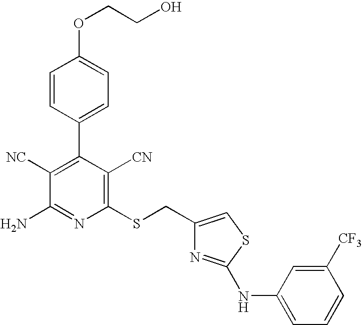

A solution of 25.4 mg (0.2 mmol) of 1,3-dichloroacetone in 0.5 ml of DMF is added to 44 mg (0.2 mmol) of 3-trifluoromethylthiourea. The reaction mixture is stirred at 80° C. for 3 h. After cooling, 62.5 mg (0.2 mmol) of 2-amino-4-[4-(2-hydroxyethoxy)phenyl]-6-mercaptopyridine-3,5-dicarbonitrile in 0.2 ml of DMF and 67 mg (0.8 mmol) of sodium bicarbonate are added. After stirring at RT overnight, the reaction mixture is filtered and purified by preparative HPLC (column: GROMSIL 120 ODS-HE-4, 5 μm, 20×50 mm; UV detection: 220 nm; injection volume: 700 μl; mobile phase A: water+0.1% formic acid, mobile phase B: acetonitrile; gradient: 0 min 10% B→1.5 min 10% B→5.5 min 90% B→7 min 90% B→7.1 min 10% B→8 min 10% B; flow rate: 25 ml/min). The product-containing fractions are concentrated under reduced pressure.

Yield: 71.6 mg (63% of theory)

LC-MS (method 2): Rₜ=2.56 min; MS (ESIpos): m/z=569 [M+H]⁺.

¹H-NMR (400 MHz, DMSO-d₆): δ=10.6 (s, 1H), 8.1 (s, 1H), 8.1 (br. s, 2H), 7.8 (d, 1H), 7.5 (m, 3H), 7.25 (d, 1H), 7.1 (m, 3H), 4.9 (t, 1H), 4.5 (s, 2H), 4.1 (t, 2H), 3.75 (q, 2H).

Analogously to example 16, the examples 17 to 28 listed in table 4 are prepared from 2-amino-4-[4-(2-hydroxyethoxy)phenyl]-6-mercaptopyridine-3,5-dicarbonitrile (examples 17 to 25) or from 2-amino-4-[4-(2-methoxyethoxy)phenyl]-6-mercaptopyridine-3,5-dicarbonitrile (preparation see WO 03/053441, example ½nd step) (examples 26 to 28):

TABLE 4

| | | MS | LC-MS | Yield |
|---|---|---|---|---|
| Example | | (ESI) | Rₜ [min] | (% of |
| No. | Structure | [M + H]⁺ | (method) | theory) |
| 17 | 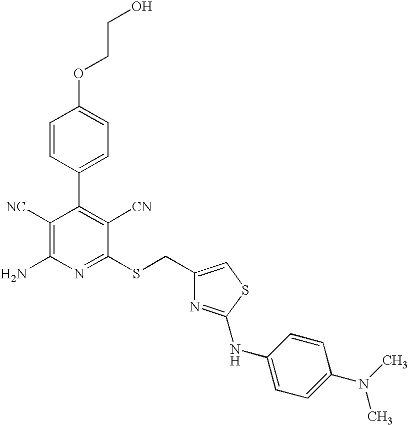 | 544 | 1.81 (2) | 29 |
| 18 | 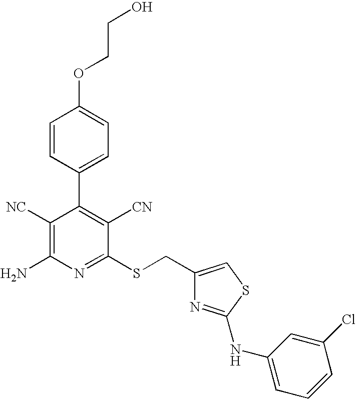 | 536 | 2.5 (2) | 65 |
| 19 | 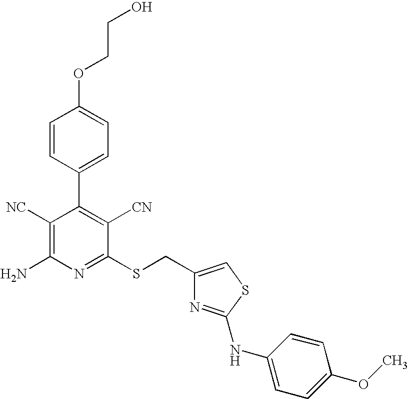 | 531 | 2.28 (2) | 65 |
| 20 | 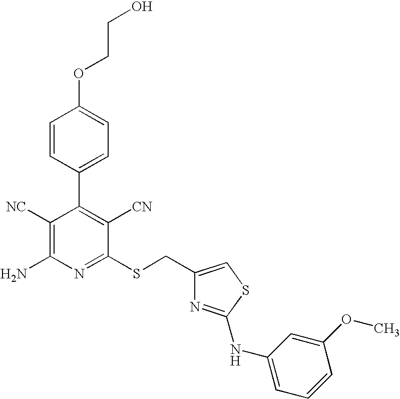 | 531 | 2.35 (2) | 76 |
| 21 | 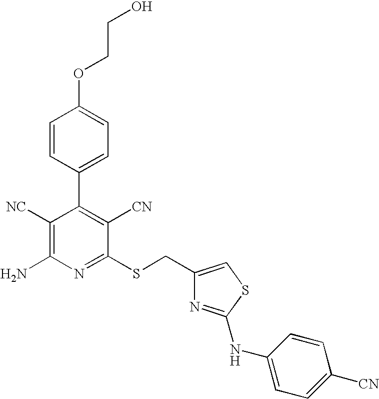 | 526 | 2.31 (2) | 32 |
| 22 | 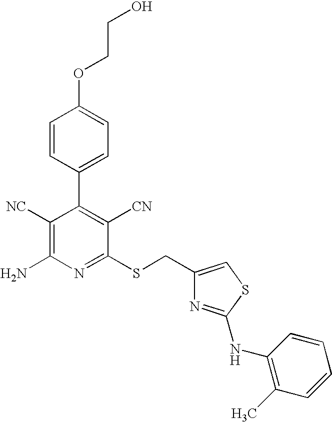 | 515 | 2.36 (2) | 77 |
| 23 | 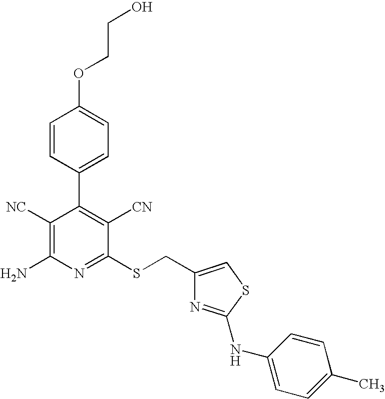 | 515 | 2.44 (2) | 64 |
| 24 | 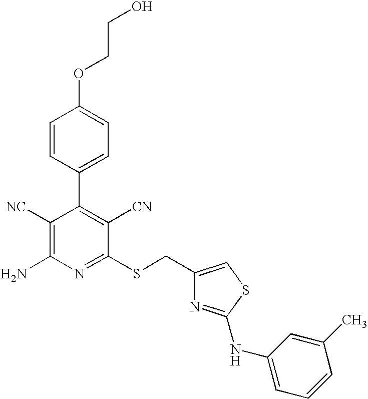 | 515 | 2.44 (2) | 78 |
| 25 | 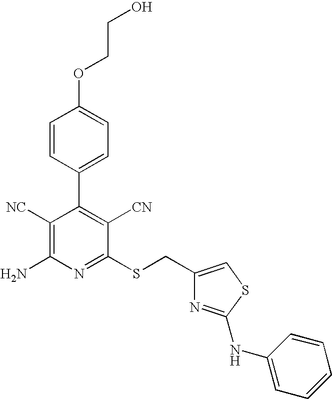 | 501 | 2.35 (2) | 70 |
| 26 | 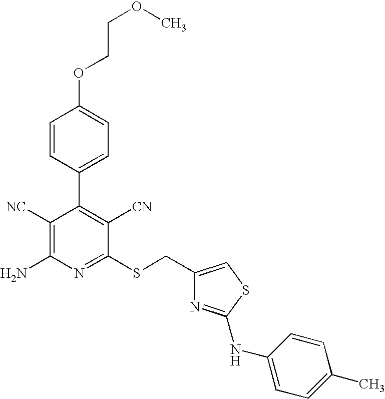 | 529 | 2.74 (4) | 27 |
| 27 | 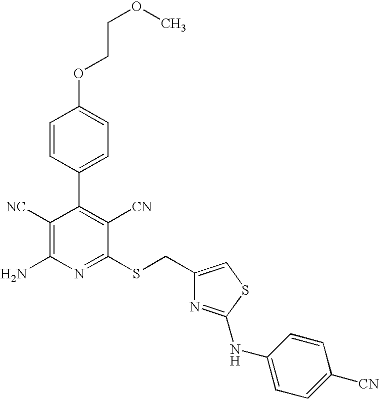 | 540 | 2.60 (4) | 51 |
| 28 | 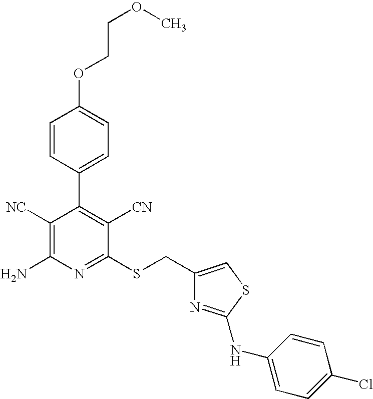 | 550 | 2.79 (2) | 12 |

Examples listed in table 5 are prepared analogously to example 1 from the corresponding starting materials:

TABLE 5

| | | MS | LC-MS | | Yield |
|---|---|---|---|---|---|
| Example | | (ESI) | Rₜ [min] | | (% of |
| No. | Structure | [M + H]⁺ | (method) | ¹H-NMR | theory) |
| 29 | 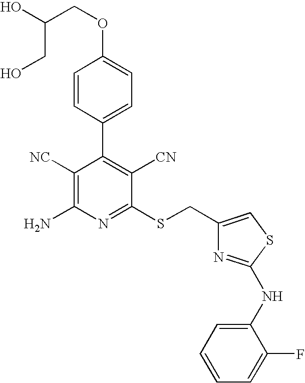 | 549 | 2.25 (4) | δ (400 MHz, DMSO-d₆) = 10.00 (s, 1H), 8.37(t, 1H), 8.07(br. s, 2H), 7.47(d, 2H), 7.23(m, 1H), 7.12 (m, 3H), 6.99(m, 2H), 4.47(s, 2H), 4.09(dd, 1H), 3.95 (dd, 1H), 3.82(m, 1H), 3.46(dd, 2H). | 68 |
| 30 | 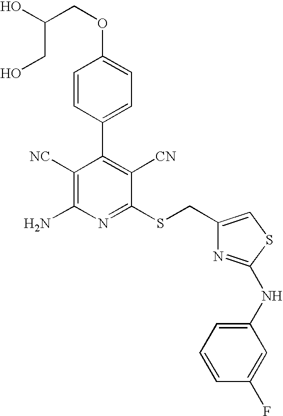 | 549 | 2.32 (4) | δ (400 MHz, DMSO-d₆) = 10.42 (s, 1H), 8.06(br. s, 2H), 7.82(s, 1H), 7.45(m, 3H), 7.30 (t, 1H), 7.10(d, 2H), 7.03(s, 1H), 6.74(dt, 1H), 4.49 (s, 2H), 4.08(dd, 1H), 3.95(dd, 1H), 3.82(m, 1H), 3.46 (dd, 2H). | 60 |
| 31 | 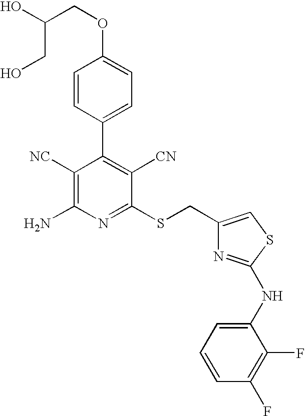 | 567 | 2.32 (4) | δ (400 MHz, DMSO-d₆) = 10.23 (s, 1H), 8.22(t, 1H), 8.07(br. s, 2H), 7.46(d, 2H), 7.09(m, 4H), 6.99 (dd, 1H), 4.99(d, 1H), 4.67(t, 1H), 4.47(s, 2H), 4.08 (dd, 1H), 3.95(dd, 1H), 3.81(m, 1H), 3.46(t, 2H). | 75 |
| 32 | 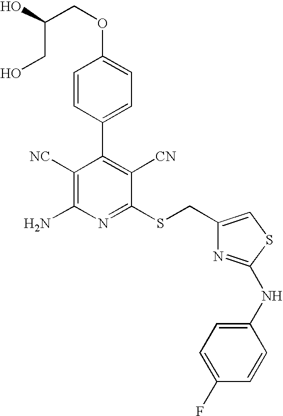 | 549 | 2.26 (4) | | 85 |
| 33 | 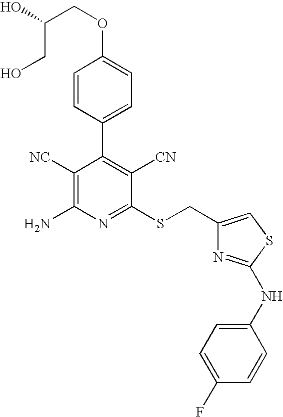 | 549 | 2.02 (3) | δ (400 MHz, DMSO-d₆) = 10.2 (br. s, 1H), 8.1(br. s, 2H), 7.6-7-65 (m, 2H), 7.47(d, 2H), 7.1-7.17(m, 4H), 6.97(s, 1H), 5.0(br. s, 1H), 4.7 (br. s, 1H), 4.44(s, 2H), 4.06-4.1(m, 1H), 3.92-3.97(m, 1H), 3.81(m, 1H), 3.46(dd, 2H). | 92 |
| 34 | 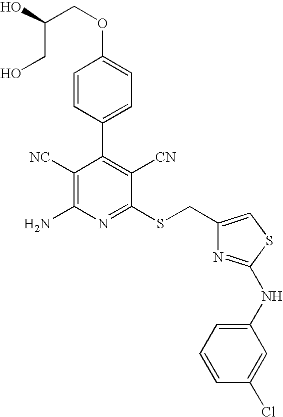 | 565 | 2.33 (2) | | 66 |
| 35 | 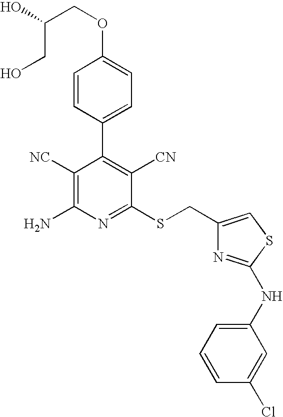 | 565 | 2.37 (4) | δ (400 MHz, DMSO-d₆) = 10.42 (s, 1H), 8.25-7.90 (br. s, 2H), 7.81(s, 1H), 7.50-7.41(m, 3H), 7.30(t, 1H), 7.10(d, 2H), 7.03 (s, 1H), 6.97(s, 1H), 5.00(d, 1H), 4.59(t, 1H), 4.49 (s, 2H), 4.08(dd, 1H), 3.95(dd, 1H), 3.86-3.78(m, 1H), 3.46(t, 2H). | 80 |
| 36 | 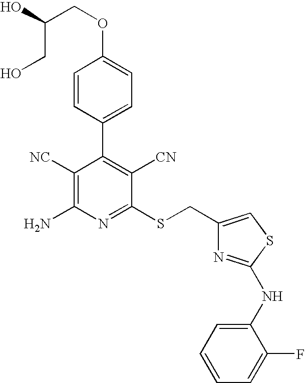 | 549 | 2.20 (2) | | 86 |
| 37 | 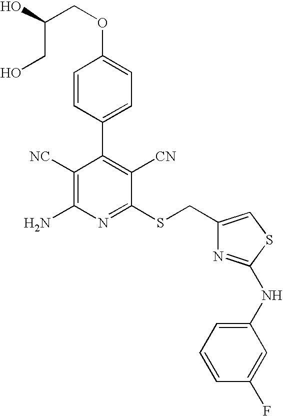 | 549 | 2.28 (4) | | 51 |
| 38 | 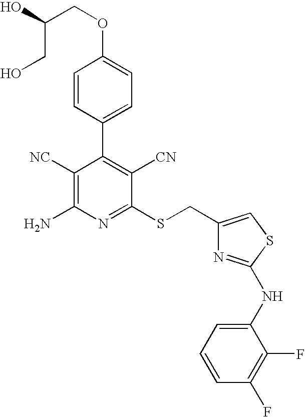 | 561 | 2.28 (4) | | 87 |
| 39 | 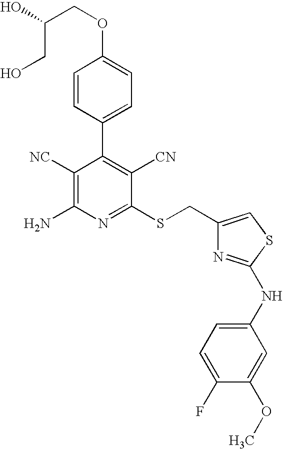 | 579 | 2.02 (3) | δ (400 MHz, DMSO-d₆) = 10.21 (s, 1H), 8.05(br. s, 2 H), 7.60(dd, 1H), 7.47(d, 2H), 7.14- 7.00(m, 4H), 6.97 (s, 1H), 4.99(d, 1H), 4.68(t, 1H), 4.47(s, 2H), 4.09 (dd, 1H), 3.94(dd, 1H), 3.86-3.78(m, 1H), 3.80(s, 3H), 3.46(t, 2H). | 48 |
| 40 | 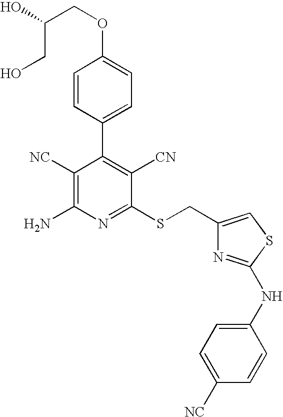 | 556 | 2.12 (2) | δ (400 MHz, DMSO-d₆) = 10.78 (s, 1H), 8.09(br. s, 2H), 7.77(dd, 2H), 7.48(d, 2H), 7.14 (s, 1H), 7.10(d, 2H), 4.99(d, 1H), 4.69(t, 1H), 4.50 (s, 2H), 4.08(dd, 1H), 3.95(dd, 1H), 3.86-3.77(m, 1H), 3.47(t, 2H). | 67 |
| 41 | 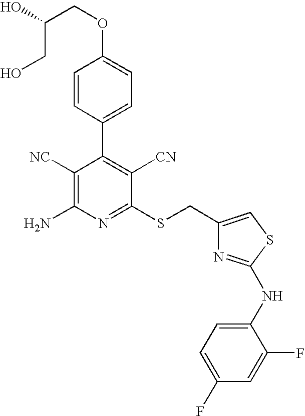 | 567 | 2.24 (2) | δ (400 Mhz, DMSO-d₆) = 9.96 (s, 1H), 8.48-8.28 (m, 1H), 8.08(br. s, 2H), 7.47(dd, 2H), 7.34-7.26(m, 1H), 7.09(d, 2H), 7.05(t, 1H), 6.99 (s, 1H), 4.99(d, 1H), 4.69(t, 1H), 4.43(s, 2H), 4.08 (dd, 1H), 3.95(dd, 1H), 3.86-3.78(m, 1H), 3.46(t, 2H). | 99 |
| 42 | 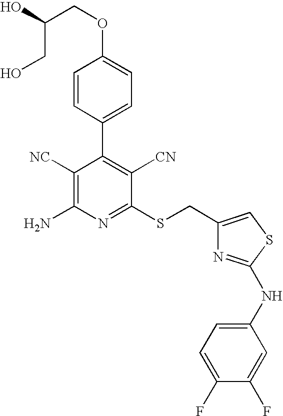 | 567 | 2.08 (3) | | 65 |
| 43 | 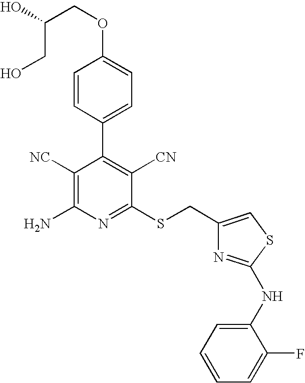 | 549 | 2.02 (3) | | 69 |
| 44 | 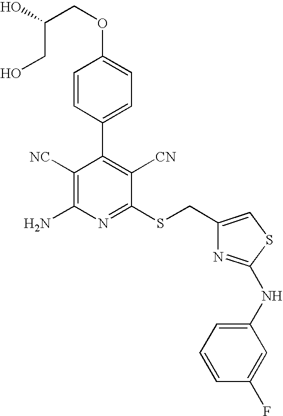 | 549 | 2.04 (3) | | 70 |
| 45 | 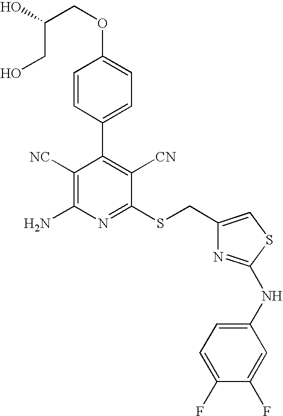 | 567 | 2.09 (3) | | 75 |
| 46 | 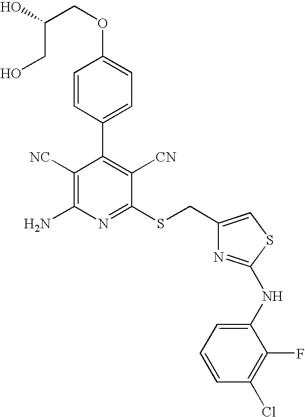 | 583 | 2.20 (3) | | 74 |
| 47 | 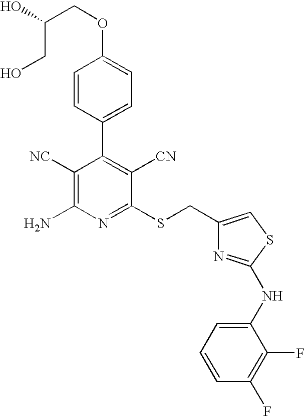 | 567 | 2.34 (4) | | 79 |
| 48 | 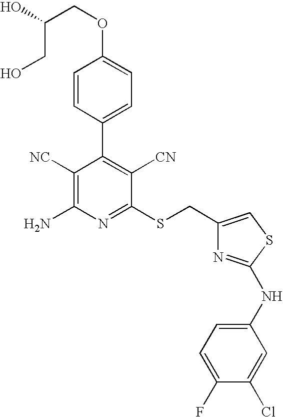 | 583 | 2.16 (3) | | 51 |
| 49 | 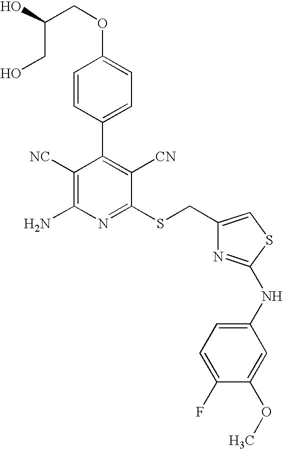 | 579 | 2.27 (4) | | 76 |
| 50 | 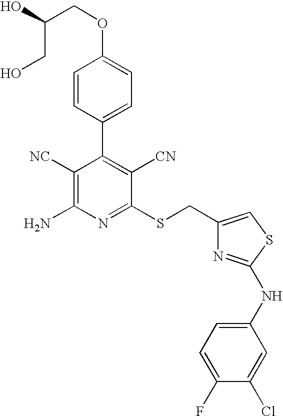 | 583 | 2.42 (4) | | 90 |
| 51 | 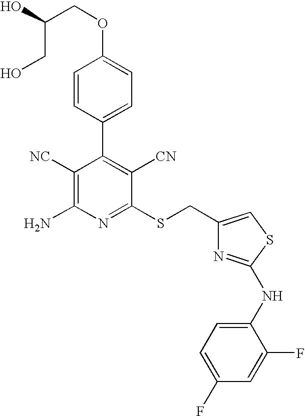 | 567 | 2.06 (3) | | 92 |
| 52 | 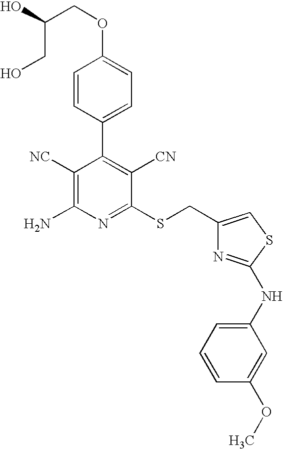 | 561 | 1.99 (3) | | 96 |
| 53 | 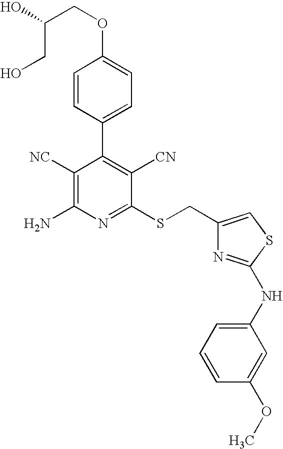 | 561 | 1.99 (3) | | 96 |
| 54 | 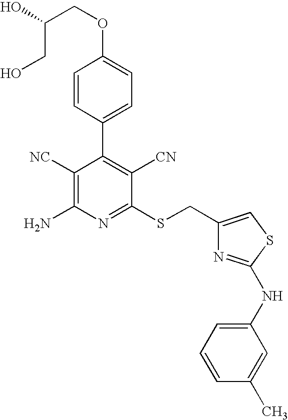 | 545 | 2.26 (2) | | 77 |
| 55 | 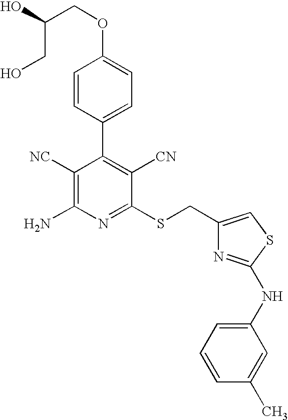 | 545 | 2.26 (2) | | 68 |
| 56 | 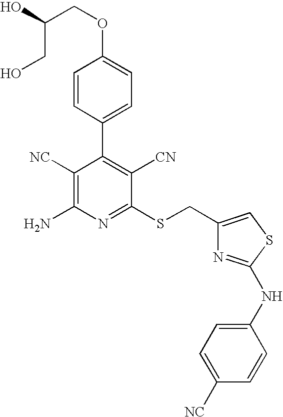 | 556 | 2.12 (2) | | 25 |

Analogously to example 16, the examples listed in table 6 are prepared from 2-amino-4-[4-(2-hydroxyethoxy)phenyl]-6-mercaptopyridine-3,5-dicarbonitrile:

TABLE 6

| | | MS | LC-MS | Yield |
|---|---|---|---|---|
| Example | | (ESI) | Rₜ [min] | (% of |
| No. | Structure | [M + H]⁺ | (method) | theory) |
| 57 | 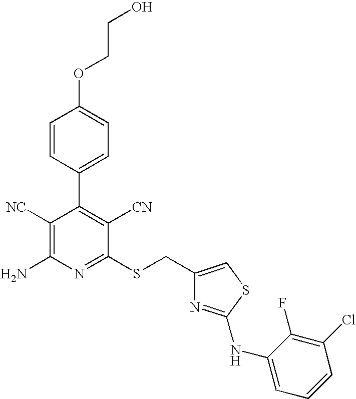 | 553 | 2.52 (2) | 68 |
| 58 | 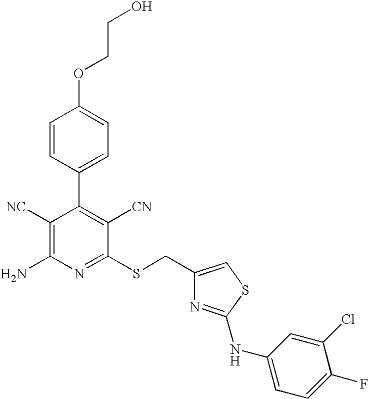 | 553 | 2.50 (2) | 42 |
| 59 | 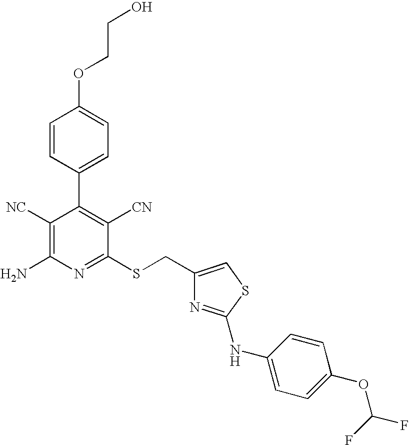 | 566 | 2.24 (3) | 20 |
| 60 | 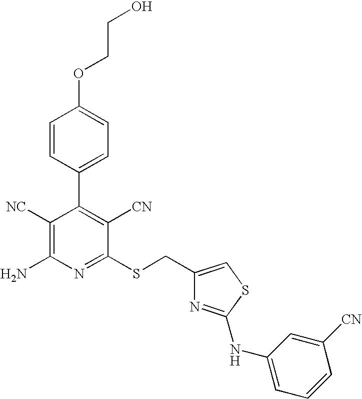 | 526 | 2.10 (3) | 83 |
| 61 | 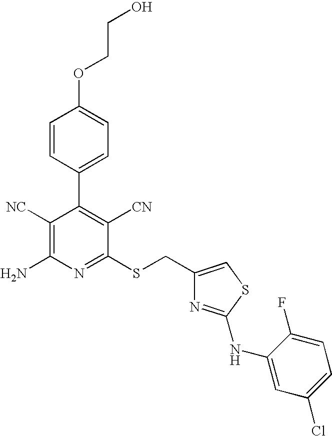 | 553 | 2.48 (2) | 70 |
| 62 | 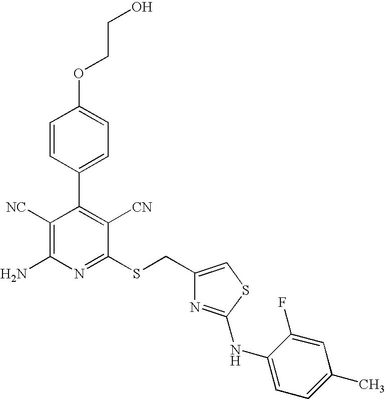 | 533 | 2.45 (2) | 70 |
| 63 | 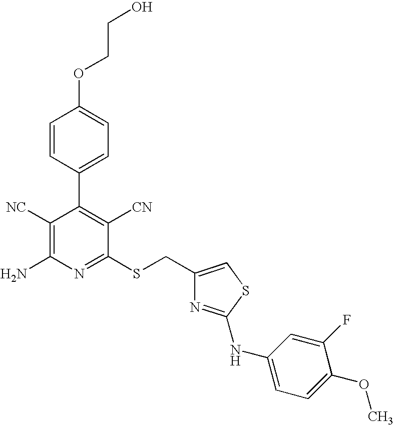 | 549 | 2.12 (3) | 66 |
| 64 | 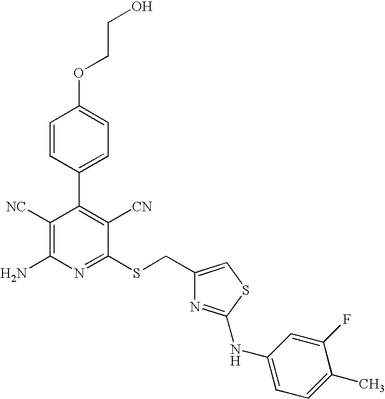 | 533 | 2.48 (2) | 54 |
| 65 | 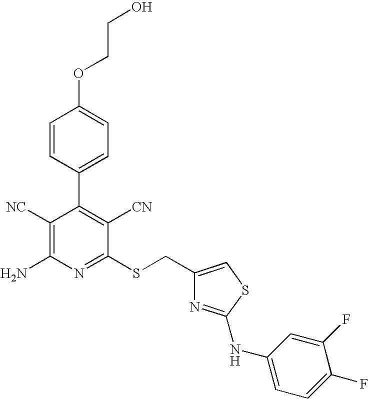 | 537 | 2.43 (2) | 34 |
| 66 | 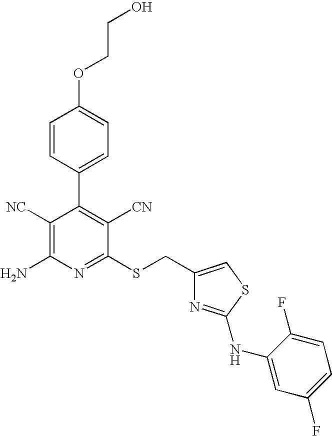 | 537 | 2.22 (3) | 16 |
| 67 | 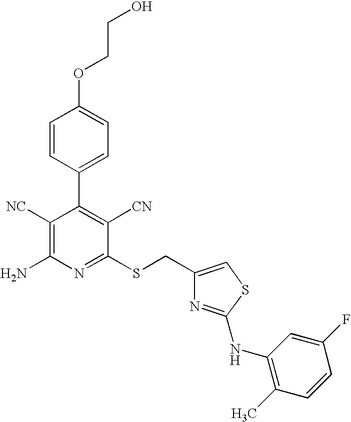 | 533 | 2.35 (2) | 20 |

### Example 68

### 2-(2-Hydroxyethoxy)amino-6-[({2-[(4-fluorophenyl)amino]-1,3-thiazol-4-yl}methyl)thio]-4-[4-(2-hydroxyethoxy)phenyl]pyridine-3,5-dicarbonitrile

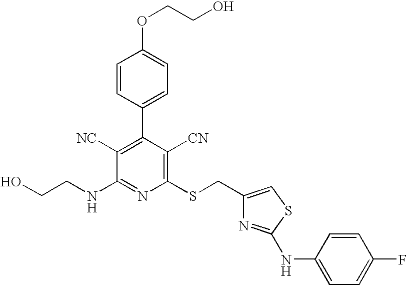

Analogously to example 8, the title compound is obtained by reacting 120 mg (0.70 mmol) of 4-fluorophenylthiourea with 89 mg (0.70 mmol) of 1,3-dichloroacetone in ethanol, followed by reaction with 245 mg (0.50 mmol) of 2-(2-hydroxyethoxy)amino-4-[4-(2-hydroxyethoxy)phenyl]-6-mercaptopyridine-3,5-dicarbonitrile (example 32A).

Yield: 30 mg (11% of theory)

¹H-NMR (400 MHz, DMSO-d₆): δ=10.24 (s, 1H), 8.03 (t, 1H), 7.61 (dd, 2H), 7.46 (d, 2H), 7.12 (m, 4H), 6.81 (s, 1H), 4.91 (t, 1H), 4.80 (t, 1H), 4.50 (s, 2H), 4.07 (t, 2H), 3.74 (dt, 2H), 3.62 (t, 2H), 3.56 (m, 2H).

LC-MS (method 3): Rₜ=2.10 min; MS (ESIpos): m/z=563 [M+H]⁺.

### Example 69

### 2-Amino-6-[({2-[(4-cyanophenyl)amino]-1,3-thiazol-4-yl}methyl)thio]-4-{4-[2-(dimethylamino)-ethoxy]phenyl}pyridine-3,5-dicarbonitrile

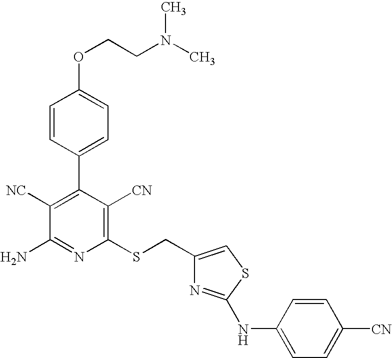

A solution of 26.6 mg (0.15 mmol) of N-(4-cyanophenyl)thiourea and 19 mg (0.15 mmol) of 1,3-dichloroacetone in 0.4 ml of DMF is stirred at +80° C. for 3 h. After cooling to RT, a solution of 50.9 mg (0.15 mmol) of the compound from example 34A in 0.2 ml of DMF and 50 mg (0.6 mmol) of sodium bicarbonate are added. The mixture is then stirred at RT for 12 h. The reaction mixture is filtered and purified directly by preparative HPLC (column: Macherey Nagel VP50/21 Nucleosil 100-5 C18 Nautilus, 5 μm, 21 mm×50 mm; wavelength: 220 nm; flow rate: 25 ml/min; mobile phase A=water+0.1% formic acid, mobile phase B=acetonitrile; gradient: 0 min 10% B→2 min 10% B→6 min 90% B→7 min 90% B→7.1 min 10% B→8 min 10% B). The product-containing fractions are combined and concentrated on a rotary evaporator.

Yield: 50 mg (57% of theory)

¹H-NMR (400 MHz, DMSO-d₆): δ=10.80 (s, 1H), 8.17 (s, 1H), 8.20-7.96 (br. s, 2H), 7.82-7.70 (m, 4H), 7.47 (d, 2H), 7.13 (d, 2H), 7.10 (s, 1H), 4.50 (s, 2H), 4.16 (t, 2H), 2.73 (t, 2H), 2.30 (s, 6H).

LC-MS (method 4): Rₜ=1.87 min; MS (ESIpos): m/z=553 [M+H]⁺.

### Example 70

### 2-Amino-4-[4-(2-aminoethoxy)phenyl]-6-[({2-[(4-fluorophenyl)amino]-1,3-thiazol-4-yl}methyl)-thio]pyridine-3,5-dicarbonitrile hydrochloride

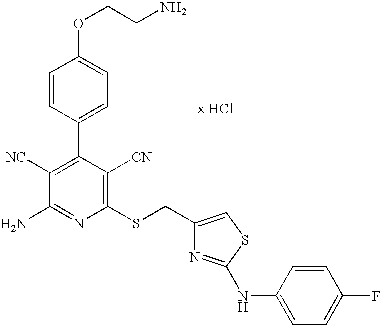

1000 mg (1.84 mmol) of the compound from example 39A are dissolved in 100 ml of dioxane, 150 mg (1.41 mmol) of palladium on activated carbon are added and the mixture is hydrogenated at 3 bar using hydrogen. After 3 h, 4 ml of 2M hydrochloric acid are added, and the mixture is hydrogenated with hydrogen at 3 bar for a further 20 h. The mixture is then filtered off with suction through Seitz clarification filters, the filter cake is washed with 50 ml of dioxane and 50 ml of toluene are added to the filtrate. After removal of the solvent on a rotary evaporator, the residue is taken up in a mixture of 50 ml of water and 50 ml of ethyl acetate. The pH is, by addition of aqueous dilute sodium bicarbonate solution, carefully adjusted to pH 9. The phases formed are separated. After drying of the organic phase over magnesium sulfate, the solvent is removed on a rotary evaporator and the residue is purified by preparative HPLC (column: YMC GEL ODS-AQ S-5/15 μm; mobile phase gradient: acetonitrile/water 10:90→95:5, with addition of 0.5% concentrated hydrochloric acid). The product-containing fractions are combined and concentrated on a rotary evaporator.

Yield: 57 mg (6% of theory)

¹H-NMR (400 MHz, DMSO-d₆): δ=10.27 (s, 1H), 8.08-7.97 (br. s, 2H), 7.67-7.59 (m, 2H), 7.51 (d, 2H), 7.20-7.09 (m, 4H), 6.98 (s, 1H), 4.47 (s, 2H), 4.25 (t, 2H), 3.31-3.21 (m, 2H).

LC-MS (method 2): Rₜ=2.08 min; MS (ESIpos): m/z=518 [M+H]⁺.

### Example 71

### 2-Amino-6-[({2-[(4-fluorophenyl)amino]-1,3-thiazol-4-yl}methyl)thio]-4-[4-(2-hydroxypropoxy)-phenyl]pyridine-3,5-dicarbonitrile

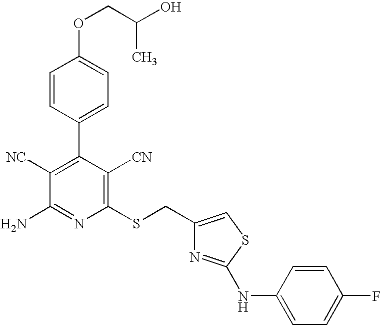

3 ml of 1M hydrochloric acid are added to a solution of 43 mg (0.06 mmol) of the compound from example 43A in 6 ml of methanol, and the mixture is stirred at RT for 20 h. 10 ml of saturated sodium bicarbonate solution are then added to the reaction mixture, and the mixture is extracted with ethyl acetate (three times 10 ml each). The combined organic phases are dried over magnesium sulfate. After removal of the solvent on a rotary evaporator, the crude product is purified chromatographically on silica gel 60 (mobile phase gradient dichloromethane/ethanol 100:1→20:1).

Yield: 0.34 g (96% of theory)

¹H-NMR (400 MHz, DMSO-d₆): δ=10.22 (s, 1H), 8.22-7.91 (br. s, 2H), 7.61 (dd, 2H), 7.47 (d, 2H), 7.18-7.06 (m, 4H), 6.97 (s, 1H), 4.91 (d, 1H), 4.46 (s, 2H), 4.02-3.94 (m, 1H), 3.94-3.83 (m, 2H), 1.17 (d, 3H).

LC-MS (method 3): Rₜ=2.26 min; MS (ESIpos): m/z=533 [M+H]⁺.

Test Descriptions:

B. Assessing the Pharmacological and Physiological Activity

The pharmacological and physiological activity of the compounds according to the invention can be demonstrated in the following assays:

Indirect Determination of the Adenosine Agonism by Way of Gene Expression

Cells of the CHO (Chinese Hamster Ovary) permanent cell line are transfected stably with the cDNA for the adenosine receptor subtypes A1, A2a and A2b. The adenosine A1 receptors are coupled to the adenylate cyclase by way of Gᵢ proteins, while the adenosine A2a and A2b receptors are coupled by way of Gₛ proteins. In correspondence with this, the formation of cAMP in the cell is inhibited or stimulated, respectively. After that, expression of the luciferase is modulated by way of a cAMP-dependent promoter. The luciferase test is optimized, with the aim of high sensitivity and reproducibility, low variance and good suitability for implementation on a robot system, by varying several test parameters, such as cell density, duration of the growth phase and the test incubation, forskolin concentration and medium composition. The following test protocol is used for pharmacologically characterizing cells and for the robot-assisted substance screening:

The stock cultures are grown, at 37° C. and under 5% CO₂, in DMEM/F12 medium containing 10% FCS (foetal calf serum) and in each case split 1:10 after 2-3 days. The test cultures are seeded in 384-well plates with 2000 cells per well and grown at 37° C. for approx. 48 hours. The medium is then replaced with a physiological sodium chloride solution (130 mM sodium chloride, 5 mM potassium chloride, 2 mM calcium chloride, 20 mM HEPES, 1 mM magnesium chloride hexahydrate, 5 mM sodium bicarbonate, pH 7.4). The substances to be tested, which are dissolved in DMSO, are pipetted into the test cultures (maximum final concentration of DMSO in the test mixture: 0.5%) in a dilution series of from 1.1×10⁻¹¹M to 3×10⁻⁶M (final concentration). 10 minutes later, forskolin is added to the A1 cells and all the cultures are subsequently incubated at 37° C. for four hours. After that, 35 μl of a solution which is composed of 50% lysis reagent (30 mM disodium hydrogenphosphate, 10% glycerol, 3% TritonX100, 25 mM TrisHCl, 2 mM dithiotreitol (DTT), pH 7.8) and 50% luciferase substrate solution (2.5 mM ATP, 0.5 mM luciferin, 0.1 mM coenzyme A, 10 mM tricine, 1.35 mM magnesium sulfate, 15 mM DTT, pH 7.8) are added to the test cultures, which are shaken for approx. 1 minute and the luciferase activity is measured using a camera system. The EC₅₀ values are determined, i.e., the concentrations at which 50% of the luciferase answer is inhibited in the case of the A1 cell, and, respectively, 50% of the maximum stimulation with the corresponding substance is achieved in the case of the A2b and A2a cells. The adenosine-analogous compound NECA (5-N-ethylcarboxamidoadenosine), which binds to all adenosine receptor subtypes with high affinity and possesses an agonistic effect, is used in these experiments as the reference compound [Klotz, K. N., Hessling, J., Hegler, J., Owman, C., Kull, B., Fredholm, B. B., Lohse, M. J., “Comparative pharmacology of human adenosine receptor subtypes—characterization of stably transfected receptors in CHO cells”, Naunyn Schmiedebergs Arch. Pharmacol., 357 (1998), 1-9).

Table 1 below lists the EC₅₀ values of representative working examples of compounds of the formula (IA) for the receptor stimulation on adenosine A1, A2a and A2b receptor subtypes:

TABLE 1

| | EC₅₀ A1 [nM] | EC₅₀ A2a | EC₅₀ A2b |
|---|---|---|---|
| Example No. | (1 μM forskolin) | [nM] | [nM] |
| 2 | 9.9 | 747 | 6.1 |
| 8 | 1 | 300 | 1 |
| 10 | 2 | >200 | 1 |
| 13 | 0.2 | 236 | 0.1 |
| 21 | 0.7 | 103 | 0.5 |
| 26 | 23 | >3000 | 74 |
| 35 | 0.4 | 142 | 0.3 |
| 39 | 0.3 | 1200 | 1.4 |
| 44 | 0.6 | 140 | 0.3 |
| 48 | 0.4 | 140 | 0.1 |
| 70 | 2.3 | >3000 | 28 |

Acute Lowering of Lipids in the Rat

Male Sprague Dawley rats (10 per group, Harlan-Netherland, 200 g) are kept without food overnight. Various dosages (1 mg/kg, 3 mg/kg, 10 mg/kg) of the substance to be tested are then administered orally to the animals. One animal group continues the test as control group without any substance administered. Before the administration of the substance and 2, 4 and 6 hours afterwards, blood samples are taken from the treated and the control animals, and EDTA plasma is obtained (500 μl of whole blood in EDTA tubes from Sarstedt, 10 min of centrifugation at 12 000 rpm). For each point in time, the content of free fatty acids and triglycerides in the plasma is determined with the aid of the automatic analysis instrument Cobas Integra 400™ from Roche Diagnosics and stated as % change compared to the value before the administration of the substance. An oral dose of acipimox (50 mg/kg, 100 mg/kg) serves as positive control.

Chronic Lowering of Lipids in the Rat

Over 24 days, male Sprague Dawley rats (15 per group, Harlan-Netherland, 200 g) are treated twice per day with various oral dosages (3 mg/kg, 10 mg/kg) of the substance to be tested. One animal group continues the test as control group without any substance administered. Before the administration of substance and on days 10 and 24 during the treatment, blood samples are taken from the unfed animals, and EDTA plasma is obtained (500 μl of whole blood in EDTA tubes from Sarstedt, 10 min of centrifugation at 12 000 rpm). For each point in time, the content of free fatty acids and triglycerides in the plasma is determined with the aid of the automatic analysis instrument Cobas Integra 400™ from Roche Diagnosics and stated as % change compared to the value before the administration of the substance. An oral dose of pioglitazone (10 mg/kg, bid) serves as positive control.

Chronic Lowering of Lipids in the Fructose-Fed Rat

For 26 days, male Sprague Dawley rats (15 per group, Harlan-Winkelmann, 180-200 g) are kept on a fructose-rich (66%) diet. After 15 days, the animals are, twice per day, treated orally with various dosages (3 mg/kg, 10 mg/kg) of the substance to be tested, for a further 10 days. One animal group continues the test as control group without any substance administered. Before the fructose diet, on day 12 of the diet (before the administration of substance) and on day 26 (10 days after the start of the treatment with substance), blood samples are taken from the animals, and EDTA plasma is obtained (500 μl of whole blood in EDTA tubes from Sarstedt, 10 min of centrifugation at 12 000 rpm). For each point in time, the content of free fatty acids and triglycerides in the plasma is determined with the aid of the automatic analysis instrument Cobas Integra 400™ from Roche Diagnosics and stated as % change compared to the value before the administration of the substance. Furthermore, the insulin concentration in the plasma is determined with the aid of the automatic analysis instrument Cobas Integra 400™ from Roche Diagnosics and stated in ng of insulin per ml of plasma.

Working Examples for Pharmaceutical Compositions

The compounds of the invention can be converted into pharmaceutical preparations in the following ways:

Tablet:

Composition:

100 mg of the compound of the invention, 50 mg of lactose (monohydrate), 50 mg of maize starch (native), 10 mg of polyvinylpyrrolidone (PVP 25) (from BASF, Ludwigshafen, Germany) and 2 mg of magnesium stearate.

Tablet weight 212 mg, diameter 8 mm, radius of curvature 12 mm.

Production:

The mixture of compound of the invention, lactose and starch is granulated with a 5% strength solution (m/m) of the PVP in water. The granules are dried and mixed with the magnesium stearate for 5 minutes. This mixture is compressed in a conventional tablet press (see above for format of the tablet). A guideline compressive force for the compression is 15 kN.

Suspension which can be Administered Orally:

Composition:

1000 mg of the compound of the invention, 1000 mg of ethanol (96%), 400 mg of Rhodigel® (xanthan gum from FMC, Pa., USA) and 99 g of water.

10 ml of oral suspension correspond to a single dose of 100 mg of the compound of the invention. Production:

The Rhodigel is suspended in ethanol, and the compound of the invention is added to the suspension. The water is added while stirring. The mixture is stirred for about 6 h until the swelling of the Rhodigel is complete.

Solution which can be Administered Orally:

Composition:

500 mg of the compound of the invention, 2.5 g of polysorbate and 97 g of polyethylene glycol 400. 20 g of oral solution correspond to a single dose of 100 mg of the compound of the invention.

Production:

The compound of the invention is suspended in the mixture of polyethylene glycol and polysorbate with stirring. The stirring process is continued until the compound of the invention has completely dissolved.

i.v. Solution:

The compound of the invention is dissolved in a concentration below the saturation solubility in a physiologically tolerated solvent (e.g. isotonic saline, 5% glucose solution and/or 30% PEG 400 solution). The solution is sterilized by filtration and used to fill sterile and pyrogen-free injection containers.

Combination with Other Drugs

The compounds according to the invention can be employed on their own or, if required, in combination with other active compounds. The present invention furthermore provides medicaments comprising at least one of the compounds according to the invention and one or more further active compounds, in particular for the treatment and/or prevention of the disorders mentioned above.

By way of example and by way of preference, the following active compounds may be mentioned as being suitable for combinations: lipid metabolism-modifying active compounds, antidiabetics (peptidic and non-peptidic), agents for treating obesity and overweight, hypotensive substances, perfusion-enhancing and/or antithrombotic agents and also antioxidants, chemokine receptor antagonists, p38 kinase inhibitors, NPY agonists, orexin agonists, anorectics, PAF-AH inhibitors, antiphlogistics (COX inhibitors, LTB₄ receptor antagonists), analgesics (aspirin), antidepressants and other psychopharmaceutics.

The present invention provides in particular combinations of at least one of the compounds
according to the invention with at least one lipid metabolism-modifying active compound, an antidiabetic (peptidic and non-peptidic), an agent for treating obesity or overweight, a hypotensive active compound and/or an antithrombotic.

The compounds according to the invention can preferably be combined with one or more
active compounds which modify lipid metabolism, by way of example and by way of preference from the group of the HMG-CoA reductase inhibitors, the inhibitors of HMG-CoA reductase expression, the squalene synthesis inhibitors, the ACAT inhibitors, the LDL receptor inductors, the cholesterol absorption inhibitors, the polymeric bile acid adsorbers, the bile acid reabsorption inhibitors, the MTP inhibitors, the lipase inhibitors, the LpL activators, fibrates, niacin, the CETP inhibitors, the PPAR-γ and/or PPAR-δ agonists, the RXR modulators, FXR modulators, the LXR modulators, the thyroid hormones and/or the thyroid mimetics, the ATP citrate lyase inhibitors, the Lp(a) antagonists, the cannabinoid receptor 1 antagonists, the leptin receptor agonists, the bombesin receptor agonists, the histamine receptor agonists and the antioxidants/radical scavengers,
antidiabetics mentioned in the Roten Liste (Red List) 2004/II, chapter 12, and also, by way of example and by way of preference, those from the group of the sulfonylureas, the biguanides, the meglitinide derivatives, the glucosidase inhibitors, the oxadiazolidinones, the thiazolidinediones, the GLP 1 receptor agonists, the glucagon antagonists, the insulin sensitizers, the CCK 1 receptor agonists, the leptin receptor agonists, the potassium channel antagonists, the inhibitors of liver enzymes involved in the stimulation of gluconeogenesis and/or glycogenolysis, the modulators of glucose uptake and the potassium channel openers, such as, for example, those disclosed in WO 97/26265 and WO 99/03861,
hypotensive active compounds, by way of example and by way of preference from the group of the calcium antagonists, the angiotensin AII antagonists, the ACE inhibitors, the beta blockers, the alpha blockers, the diuretics, the phosphodiesterase inhibitors, the sGC stimulators, the cGMP enchancers, the aldosterone antagonists, the mineralocorticoid receptor antagonists, the ECE inhibitors and the vasopeptidase inhibitors, and/or
agents having antithrombotic action, by way of example and by way of preference from the group of the platelet aggregation inhibitors or the anticoagulants.

The lipid metabolism-modifying active compounds are to be understood as meaning, by way of preference, compounds from the group of the HMG-CoA reductase inhibitors, the squalene synthesis inhibitors, the ACAT inhibitors, the cholesterol absorption inhibitors, the MTP inhibitors, the lipase inhibitors, the thyroid hormones and/or thyroid mimetics, the niacin receptor agonists, the CETP inhibitors, the PPAR-gamma agonists, the PPAR-delta agonists, the polymeric bile acid adsorbers, the bile acid reabsorption inhibitors, the antioxidants/radical scavengers and the cannabinoid receptor 1 antagonists.

In a preferred embodiment of the invention, the compounds according to the invention are administered in combination with an HMG-CoA reductase inhibitor from the class of the statins, such as, by way of example and by way of preference, lovastatin, simvastatin, pravastatin, fluvastatin, atorvastatin, rosuvastatin, cerivastatin or pitavastatin.

In a preferred embodiment of the invention, the compounds according to the invention are administered in combination with a squalene synthesis inhibitor, such as, by way of example and by way of preference, BMS-188494 or TAK-475.

In a preferred embodiment of the invention, the compounds according to the invention are administered in combination with an ACAT inhibitor, such as, by way of example and by way of preference, melinamide, pactimibe, eflucimibe or SMP-797.

In a preferred embodiment of the invention, the compounds according to the invention are administered in combination with a cholesterol absorption inhibitor, such as, by way of example and by way of preference, ezetimibe, tiqueside or pamaqueside.

In a preferred embodiment of the invention, the compounds according to the invention are administered in combination with an MTP inhibitor, such as, by way of example and by way of preference, implitapide or JTT-130.

In a preferred embodiment of the invention, the compounds according to the invention are administered in combination with a lipase inhibitor, such as, by way of example and by way of preference, orlistat.

In a preferred embodiment of the invention, the compounds according to the invention are administered in combination with a thyroid hormone and/or thyroid mimetic, such as, by way of example and by way of preference, D-thyroxine or 3,5,3′-triiodothyronine (T3).

In a preferred embodiment of the invention, the compounds according to the invention are administered in combination with an agonist of the niacin receptor, such as, by way of example and by way of preference, niacin, acipimox, acifran or radecol.

In a preferred embodiment of the invention, the compounds according to the invention are administered in combination with a CETP inhibitor, such as, by way of example and by way of preference, torcetrapib, JTT-705 or CETP vaccine (Avant).

In a preferred embodiment of the invention, the compounds according to the invention are administered in combination with a PPAR-gamma agonist, such as, by way of example and by way of preference, pioglitazone or rosiglitazone.

In a preferred embodiment of the invention, the compounds according to the invention are administered in combination with a PPAR-delta agonist, such as, by way of example and by way of preference, GW-501516.

In a preferred embodiment of the invention, the compounds according to the invention are administered in combination with a polymeric bile acid adsorber, such as, by way of example and by way of preference, cholestyramine, colestipol, colesolvam, CholestaGel or colestimide.

In a preferred embodiment of the invention, the compounds according to the invention are administered in combination with a bile acid reabsorption inhibitor, such as, by way of example and by way of preference, ASBT (=IBAT) inhibitors, such as, for example, AZD-7806, S-8921, AK-105, BARI-1741, SC-435 or SC-635.

In a preferred embodiment of the invention, the compounds according to the invention are administered in combination with an antioxidant/radical scavenger, such as, by way of example and by way of preference, probucol, AGI-1067, BO-653 or AEOL-10150.

In a preferred embodiment of the invention, the compounds according to the invention are administered in combination with a cannabinoid receptor 1 antagonist, such as, by way of example and by way of preference, rimonabant or SR-147778.

Antidiabetics are to be understood as meaning, by way of preference, insulin and insulin derivatives, and also oral hypoglycemics. Here, insulin and insulin derivatives include both insulins of animal, human or biotechnological origin and mixtures thereof. The oral hypoglycemics preferably include sulfonylureas, biguanids, meglitinide derivatives, glucosidase inhibitors and PPAR-gamma agonists.

In a preferred embodiment of the invention, the compounds according to the invention are administered in combination with insulin.

In a preferred embodiment of the invention, the compounds according to the invention are administered in combination with a sulfonylurea, such as, by way of example and by way of preference, tolbutamide, glibenclamide, glimepiride, glipizide or gliclazide.

In a preferred embodiment of the invention, the compounds according to the invention are administered in combination with a biguanide, such as, by way of example and by way of preference, metformin.

In a preferred embodiment of the invention, the compounds according to the invention are administered in combination with a meglitinide derivative, such as, by way of example and by way of preference, repaglinide or nateglinide.

In a preferred embodiment of the invention, the compounds according to the invention are administered in combination with a glucosidase inhibitor, such as, by way of example and by way of preference, miglitol or acarbose.

In a preferred embodiment of the invention, the compounds according to the invention are administered in combination with a PPAR-gamma agonist, for example from the class of the thiazolidindiones, such as, by way of example and by way of preference, pioglitazone or rosiglitazone.

Hypotensive agents are to be understood as meaning, by way of preference, compounds from the group of the calcium antagonists, the angiotensin All antagonists, the ACE inhibitors, the beta blockers, the alpha blockers and the diuretics.

In a preferred embodiment of the invention, the compounds according to the invention are administered in combination with a calcium antagonist, such as, by way of example and by way of preference, nifedipin, amlodipin, verapamil or diltiazem.

In a preferred embodiment of the invention, the compounds according to the invention are administered in combination with an angiotensin AII antagonist, such as, by way of example and by way of preference, losartan, valsartan, candesartan, embusartan, olmesartan or telmisartan.

In a preferred embodiment of the invention, the compounds according to the invention are administered in combination with an ACE inhibitor, such as, by way of example and by way of preference, enalapril, captopril, ramipril, delapril, fosinopril, quinopril, perindopril or trandopril.

In a preferred embodiment of the invention, the compounds according to the invention are administered in combination with a beta blocker, such as, by way of example and by way of preference, propranolol, atenolol, timolol, pindolol, alprenolol, oxprenolol, penbutolol, bupranolol, metipranolol, nadolol, mepindolol, carazalol, sotalol, metoprolol, betaxolol, celiprolol, bisoprolol, carteolol, esmolol, labetalol, carvedilol, adaprolol, landiolol, nebivolol, epanolol or bucindolol.

In a preferred embodiment of the invention, the compounds according to the invention are administered in combination with an alpha blocker, such as, by way of example and by way of preference, prazosin.

In a preferred embodiment of the invention, the compounds according to the invention are administered in combination with a diuretic, such as, by way of example and by way of preference, furosemide.

In a preferred embodiment of the invention, the compounds according to the invention are administered in combination with antisympathotonics, such as reserpine, clonidine or alpha-methyldopa, with potassium channel agonists, such as minoxidil, diazoxide, dihydralazine or hydralazine, or with nitric oxide-releasing substances, such as glycerol nitrate or nitroprusside sodium.

Agents having antithrombotic action are to be understood as meaning, by way of preference, compounds from the group of the platelet aggregation inhibitors or the anticoagulants.

In a preferred embodiment of the invention, the compounds according to the invention are administered in combination with a platelet aggregation inhibitor, such as, by way of example and by way of preference, aspirin, clopidogrel, ticlopidine or dipyridamol.

In a preferred embodiment of the invention, the compounds according to the invention are administered in combination with a thrombin inhibitor, such as, by way of example and by way of preference, ximelagatran, melagatran, bivalirudin or clexane.

In a preferred embodiment of the invention, the compounds according to the invention are administered in combination with a GPIIb/IIIa antagonist, such as, by way of example and by way of preference, tirofiban or abciximab.

In a preferred embodiment of the invention, the compounds according to the invention are administered in combination with a factor Xa inhibitor, such as, by way of example and by way of preference, DX-9065a, DPC 906, JTV 803, BAY 59-7939, DU-176b, fidexaban, razaxaban, fondaparinux, idraparinux, PMD-3112, YM-150, KFA-1982, EMD-503982, MCM-17, MLN-1021, SSR-126512 or SSR-128428.

In a preferred embodiment of the invention, the compounds according to the invention are administered in combination with heparin or a low molecular weight (LMW) heparin derivative.

In a preferred embodiment of the invention, the compounds according to the invention are administered in combination with a vitamin K antagonist, such as, by way of example and by way of preference, coumarin.

1. A method for treating dyslipidemia metabolic syndrome or diabetes, comprising administering a pharmaceutically acceptable amount of a compound of formula (IA): 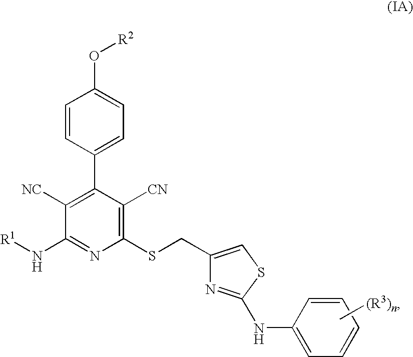
in which
R¹ represents hydrogen or represents (C₁-C₆)-alkyl which may be substituted by hydroxyl, amino, mono- or di-(C₁-C₄)-alkylamino, pyrrolidino, piperidino, morpholino, piperazino or N′-methylpiperazino,
R² represents (C₂-C₆)-alkyl, which is mono- or di-substituted by identical or different substituents selected from the group consisting of hydroxyl, (C₁-C₄)-alkoxy, amino, mono- and di-(C₁-C₄)-alkylamino,
R³ represents a substituent selected from the group consisting of halogen, cyano, nitro, (C₁-C₆)-alkyl, hydroxyl, (C₁-C₆)-alkoxy, amino, mono- and di-(C₁-C₆)-alkylamino, carboxyl and (C₁-C₆)-alkoxycarbonyl,
where alkyl and alkoxy for their part may each be substituted up to five times by fluorine,
and
n represents the number 0, 1, 2, 3, 4 or 5,
where, if the substituent R³ is present more than once, its meanings may be identical or different,
and their salts, solvates and solvates of the salts thereof.

2. The method according to claim 1, wherein
R¹ represents hydrogen or represents (C₁-C₄)-alkyl, which may be substituted by hydroxyl, amino or dimethylamino,
R² represents (C₂-C₄)-alkyl, which is mono- or disubstituted by identical or different substituents selected from the group consisting of hydroxyl, methoxy and amino,
R³ represents a substituent selected from the group consisting of halogen, cyano, nitro, (C₁-C₄)-alkyl, hydroxyl, (C₁-C₄)-alkoxy, amino, mono- and di-(C₁-C₄)-alkylamino, carboxyl and (C₁-C₄)-alkoxycarbonyl,
where alkyl and alkoxy for their part may each be substituted up to three times by fluorine,
and
n represents the number 0, 1 or 2,
where, if the substituent R³ is present twice, its meanings may be identical or different, and their salts, solvates and solvates thereof.

3. The method according to claim 1, wherein
R¹ represents hydrogen,
R² represents ethyl, n-propyl or isopropyl, which are in each case mono- or disubstituted by identical or different substituents selected from the group consisting of hydroxyl, methoxy and amino,
R³ represents a substituent selected from the group consisting of fluorine, chlorine, bromine, cyano, nitro, methyl, ethyl, trifluoromethyl, hydroxyl, methoxy, ethoxy, amino, mono- and dimethylamino, carboxyl, methoxycarbonyl and ethoxycarbonyl,
and
n represents the number 0, 1 or 2,
where, if the substituent R³ is present twice, its meanings may be identical or different, and their salts, solvates and solvates of the salts thereof.

4. A method for treating dyslipidemia, metabolic syndrome or diabetes, comprising administering a pharmaceutically acceptable amount of a compound of formula (IB): 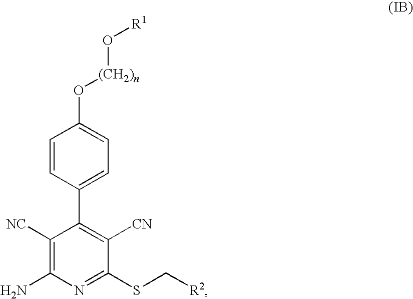
in which
n represents a number 2, 3 or 4,
R¹ represents hydrogen or (C₁-C₄)-alkyl
and
R₂ represents pyridyl or thiazolyl, which for its part may be substituted by (C₁-C₄)-alkyl, halogen, amino, dimethylamino, acetylamino, guanidino, pyridylamino, thienyl, furyl, imidazolyl, pyridyl, morpholinyl, thiomorpholinyl, piperidinyl, piperazinyl, N—(C₁-C₄)-alkylpiperazinyl, pyrrolidinyl, oxazolyl, isoxazolyl, pyrimidinyl, pyrazinyl, optionally (C₁-C₄)-alkyl-substituted thiazolyl or phenyl which is optionally substituted up to three times by halogen, (C₁-C₄)-alkyl or (C₁-C₄)-alkoxy,
and their salts, hydrates, hydrates of the salts and solvates thereof.

5. The method according to claim 4, wherein
n represents the number 2,
R¹ represents hydrogen, methyl or ethyl
and
R² represents pyridyl or thiazolyl which for its part may be substituted by methyl, ethyl, fluorine, chlorine, amino, dimethylamino, acetylamino, guanidino, 2-pyridylamino, 4-pyridylamino, thienyl, pyridyl, morpholinyl, piperidinyl, optionally methyl-substituted thiazolyl or phenyl which is optionally substituted up to three times by chlorine or methoxy,
and their salts, hydrates, hydrates of the salts and solvates thereof.

6. The method according to claim 4, wherein R¹ represents hydrogen or methyl.

7. The method according to claim 4, wherein
n represents the number 2,
R¹ represents hydrogen or methyl
and
R² represents pyridyl or thiazolyl, which for its part may be substituted by methyl, chlorine, amino, dimethylamino, acetylamino, guanidino, 2-pyridylamino, 4-pyridylamino, thienyl, pyridyl, morpholinyl, 2-methylthiazol-5-yl, phenyl, 4-chlorophenyl or 3,4,5-trimethoxyphenyl,
and their salts, hydrates, hydrates of the salts and solvates thereof.

8. A method for treating dyslipidemia, metabolic syndrome or diabetes, comprising administering a pharmaceutically acceptable amount of a compound of: 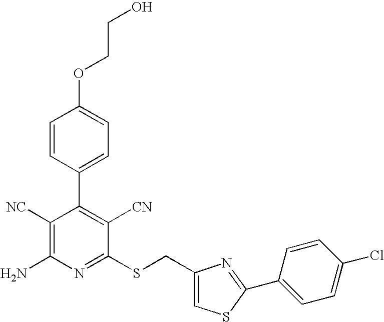
and its salts, hydrates, hydrates of the salts and solvates thereof.

9. A method for treating dyslipidemia, metabolic syndrome or diabetes, comprising administering a pharmaceutically acceptable amount of a pharmaceutical composition comprising a compound of formula (IA) as defined in claim 1 and a compound of formula (IB) as defined in claim 4.

10. A method for treating dyslipidemia, metabolic syndrome or diabetes in association with hypertension and cardiovascular disorders, comprising administering a pharmaceutically acceptable amount of a pharmaceutical composition comprising a compound of formula (IA) as defined in claim 1 and a compound of formula (IB) as defined in claim 4.

11. A pharmaceutical composition for treating dyslipidemia, metabolic syndrome and diabetes, comprising a compound of formula (IA) as defined in claim 1 and a compound of formula (IB) as defined in claim 4, and a pharmaceutically acceptable carrier.

12. A pharmaceutical composition for treating dyslipidemia, metabolic syndrome and diabetes in association with hypertension and cardiovascular disorders, comprising a compound of formula (IA) as defined in claim 1 and a compound of formula (IB) as defined in claim 4 and a pharmaceutically acceptable carrier.

13. A pharmaceutical composition for treating dyslipidemia, metabolic syndrome and diabetes comprising a compound of formula (IA) of claim 1 or formula (IB) of claim 4, in combination with a further active compound, selected from the group consisting of, a anti-hypertensive drug, an aldosterone antagonist, a renin inhibitor, a mineralcorticoid receptor antagonists, and a lipid metabolism-modifying active compound.

14. A method for the treatment and/or prevention of dyslipidemia, metabolic syndrome and diabetes as single illness and in association with hypertension and cardiovascular disorders in humans and animals by administering an effective amount of at least one compound as defined in claim 1, or a pharmaceutical composition as defined in claim 11.

15. The pharmaceutical composition of claim 13, wherein the anti-hypertensive drug is a betablocker, calcium antagonist, diuretic, ACE inhibitor, AT1 antagonist or nitrate.

16. The pharmaceutical composition of claim 13, wherein the lipid metabolism-modifying active compound is an HMG CoA reductase inhibitor, PPAR-gamma agonist, PPAR-delta agonist, fibrate, niacin, or CETP inhibitor.
